(19) United States
(12) Patent Application Publication
(10) Pub. No.: US 2026/0130833 A1
(43) Pub. Date: May 14, 2026

(54) METAL-ORGANIC FRAMEWORKS AND INCORPORATION OF HYDROPHOBIC ADSORBATES THEREIN

(71) Applicant: ELC Management LLC, Melville, NY (US)

(72) Inventors: John Carl HADYNSKI, Cincinnati, OH (US); Adarsh NAYARASSERY-NARAYANAN, Richardson, TX (US); Mario WRIEDT, Wylie, TX (US); Snehal M. SHAH, Nesconset, NY (US); John Robert DOINA, Ronkonkoma, NY (US)

(73) Assignee: ELC Management LLC, Melville, NY (US)

(21) Appl. No.: 18/941,649

(22) Filed: Nov. 8, 2024

(51) Int. Cl. A61K 8/58; A61K 8/35; A61K 8/37; A61Q 17/04; B01J 20/22; B01J 20/28; C07F 3/06

(52) U.S. Cl. CPC A61K 8/58; A61K 8/35; A61K 8/37; A61Q 17/04; B01J 20/226; B01J 20/28004; B01J 20/28007; B01J 20/28064; B01J 20/28066; B01J 20/2808; B01J 20/28083; C07F 3/06

(57) ABSTRACT

Metal-organic frameworks (MOFs) may be prepared to contain a high loading of one or more hydrophobic adsorbates therein. Suitable metal-organic frameworks may exhibit stable retention of hydrophobic adsorbates within the pore space of the MOF, such as MOF loadings of at least 20% without appreciable environmental leaching over 24 hours. Certain hydrophobic adsorbates may exhibit unexpectedly higher photostability and UV absorption as compared to the respective free hydrophobic adsorbates.

### FIELD

The present disclosure generally relates to metal-organic frameworks (MOFs) and, more particularly, metal-organic frameworks having high stability, known or potential biocompatibility, and suitable hydrophobicity, pore size and surface area values for stable and high-loading incorporation of active materials (adsorbates) therein.

### BACKGROUND

Metal-organic frameworks (MOFs) are a relatively new class of highly porous materials with potential applications in a wide range of fields. MOFs comprise multidentate organic ligands that function as “struts” or “scaffolding” that bridge between metal atoms or clusters of metal atoms, which collectively define an extended coordination structure having uniform pores defined therein. The pore size and pore distribution of MOFs is typically defined by the molecular size and ligating topology of the organic ligands, as well as the coordination geometry of the metal atom(s) defining the MOF structure. The pore size and pore distribution of MOFs may be tuned through selection of the multidentate organic ligands and the metal atom(s) (metal ions or metal clusters) used to construct the MOF. Because organic ligands may be readily modified, MOFs as a whole exhibit a great breadth of structural diversity (e.g., different cage structures), much more so than that found for other porous materials, such as zeolites. A given cage topology may be realized with several related organic ligands and/or different metal ions, but the size and distribution of the resulting pores may differ somewhat depending on the specific organic ligands and metal ions used to construct the MOF. In some cases, even small changes in structure of the organic ligand and/or the metal may result in MOFs having various and differing topologies, including different crystalline phases.

By virtue of their highly porous character, MOFs may incorporate a variety of molecules (adsorbates) within their pore structure. Factors governing whether a particular adsorbate will be incorporated within the pores of a given MOF may include, for example, hydrophobicity or hydrophilicity of the adsorbate in comparison to that of the pores, the molecular size of the adsorbate with respect to the size and geometry of the pores, and ability of the adsorbate to access the pores. Among the areas that have been actively investigated for use of MOFs include, for example, gas storage, separations, sensing, catalysis, drug delivery, and waste remediation, among others. High costs and low loading of adsorbates within the pore space of the MOF are among the challenges that have hampered more widespread utilization of these materials.

### SUMMARY

In some embodiments, the present disclosure provides metal organic frameworks comprising hydrophobic cavities having a pore diameter of about 1 nm to about 2 nm, and, optionally, at least one adsorbate within the hydrophobic cavities. In various aspects, compositions may comprise: a metal-organic framework having a plurality of pores defined therein; and at least one hydrophobic adsorbate incorporated within the plurality of pores. The at least one hydrophobic adsorbate is present within the plurality of pores in an amount ranging from about 20 wt % to about 50 wt %, based upon mass of the metal-organic framework, and photodegradation of the hydrophobic adsorbate, when incorporated within the plurality of pores, is at least partially suppressed relative to free hydrophobic adsorbate not incorporated within the plurality of pores. Personal care products may comprise such compositions.

In some or other embodiments, the present disclosure provides cyclodextrin-based metal-organic frameworks comprising a cyclodextrin defining a plurality of pores having a pore size of at least about 1 nm in diameter, and, optionally, at least one adsorbate within the plurality of pores. In various aspects, compositions may comprise: a cyclodextrin-based metal-organic framework comprising a cyclodextrin defining a plurality of pores therein, and at least one hydrophobic adsorbate incorporated within the plurality of pores. The at least one hydrophobic adsorbate is present within the plurality of pores in an amount ranging from about 20 wt % to about 35 wt %, based upon mass of the metal-organic framework, and photodegradation of the hydrophobic adsorbate, when incorporated within the plurality of pores, is at least partially suppressed relative to free hydrophobic adsorbate not incorporated within the plurality of pores. Personal care products may comprise such compositions.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, as will occur to those skilled in the art and having the benefit of this disclosure.

FIG. 1 is an X-Ray powder diffraction pattern of various particle sizes of AVB@MAF-6.

FIG. 2A shows overlaid UV-VIS spectra of free avobenzone dissolved in methanol and irradiated for various times at 365 nm using a 100 W lamp located 14 cm from the sample. FIG. 2B shows overlaid UV-VIS spectra of AVB@MAF-6 (average particle size=564 nm) dispersed in methanol and irradiated for various times at 365 nm using a 100 W lamp located 14 cm from the sample.

FIG. 3 shows overlaid UV-VIS spectra of AVB@MAF-6 dispersed in methanol and having an average particle size of 317 nm irradiated for various times at 365 nm using a 100 W lamp located 14 cm from the sample.

FIG. 4 shows overlaid UV-VIS spectra of AVB@MAF-6 dispersed in methanol and having various average particle sizes in comparison to free avobenzone.

FIG. 5A shows overlaid UV-VIS spectra of free avobenzone layered as a thin-film in a lotion base and irradiated for various times at 365 nm using a 100 W lamp located 14 cm from the sample. FIG. 5B shows overlaid UV-VIS spectra of AVB@MAF-6 layered as a thin-film in a lotion base and irradiated for various times at 365 nm using a 100 W lamp located 14 cm from the sample.

FIG. 6 shows overlaid UV-VIS spectra of OMC@MAF-6 dispersed in methanol and irradiated for various times at 365 nm using a 100 W lamp located 14 cm from the sample.

FIG. 7 shows overlaid UV-VIS spectra of the supernatant liquids at various pH values obtained from AVB@MAF-6 in comparison to free avobenzone.

FIG. 8 is an X-Ray powder diffraction pattern of β-CD-K-MOF synthesized using the alternative sources of potassium ions.

FIG. 9 is an X-Ray powder diffraction pattern of AVB@β-CD-K-MOF prepared under various conditions.

FIG. 10 is an X-Ray powder diffraction pattern of OMC@)β-CD-K-MOF prepared under various solvent-assisted conditions.

FIG. 11 shows overlaid UV-VIS spectra of AVB@β-CD-K-MOF dispersed in methanol and irradiated at 365 nm for various irradiation times using a 100 W lamp located 14 cm from the sample.

FIG. 12 shows overlaid UV-VIS spectra of AVB@β-CD-K-MOF having an average particle size of 269 nm dispersed in methanol and irradiated for various times at 365 nm using a 100 W lamp located 14 cm from the sample.

FIG. 13 shows overlaid UV-VIS spectra for AVB@β-CD-K-MOF and free avobenzone at equivalent avobenzone concentrations.

FIG. 14 shows overlaid UV-VIS spectra of AVB@β-CD-K-MOF layered as a thin-film in a lotion base and irradiated for various times at 365 nm using a 100 W lamp located 14 cm from the sample.

FIG. 15 shows X-Ray powder diffraction patterns of the AVB@β-CD-K-MOF treated at the various pH values.

FIG. 16 is an X-Ray powder diffraction pattern of HP-β-CD-K-MOF.

FIG. 17 shows overlaid UV-VIS spectra of AVB@HP-β-CD-K-MOF dispersed in methanol and irradiated for various irradiation times at 365 nm using a 100 W lamp located 14 cm from the sample.

FIG. 18 shows overlaid UV-VIS spectra of AVB@HP-β-CD-K-MOF layered as a thin-film in a lotion base and irradiated for various times at 365 nm using a 100 W lamp located 14 cm from the sample.

FIG. 19 shows overlaid X-Ray powder diffraction spectra for γ-CD-K-MOF and AVB@γ-CD-K-MOF.

FIG. 20 shows overlaid UV-VIS spectra of AVB@γ-CD-K-MOF layered as a thin-film in a lotion base and irradiated for various times at 365 nm using a 100 W lamp located 14 cm from the sample.

FIG. 21 shows overlaid UV-VIS spectra of AVB@ZIF-8 layered as a thin-film in a lotion base and irradiated for various times at 365 nm using a 100 W lamp located 14 cm from the sample.

FIG. 22 shows overlaid UV-VIS spectra of AVB@MIL-100(Fe) layered as a thin-film in a lotion base and irradiated for various times at 365 nm using a 100 W lamp located 14 cm from the sample.

FIG. 23 shows overlaid UV-VIS spectra of AVB@UiO-66-NH₂ layered as a thin-film in a lotion base and irradiated for various times at 365 nm using a 100 W lamp located 14 cm from the sample.

### DETAILED DESCRIPTION

The present disclosure generally relates to metal-organic frameworks (MOFs) and, more particularly, metal-organic frameworks having high stability, known or potential biocompatibility, and suitable hydrophobicity, pore size and surface area values for stable and high-loading incorporation of active materials (adsorbates) therein.

The present disclosure provides metal-organic frameworks that are capable of incorporating high loadings of adsorbates within their pore structure. Example adsorbates are not believed to be particularly limited, but adsorption of ultraviolet-absorbing molecules (UV absorbers) may be particularly advantageous, as discussed hereinafter. Sunscreens and other personal care products may be formulated containing the metal-organic frameworks loaded with such UV-absorbers. The term “UV filter” may be used synonymously with “UV absorber” in the case of sunscreens and other personal care products.

In the personal care space, UV absorbers may be used for absorbing harmful UV radiation and may be present in sunscreen formulations for protecting a wearer's skin. While effective, the effective lifetime of sunscreen formulations is limited once applied to a wearer's skin and exposed to the sun. One reason for the limited effective lifetime of sunscreens is that they tend to “wear off” the skin due to activities and processes such as abrasion, perspiration, immersion, and the like. Physical loss of the sunscreen and its UV absorbers may be mitigated to some degree through choice of the base materials in which the UV absorbers are formulated. For example, the base materials may be tailored to provide effective dispersion of the UV absorbers and maintain extended adherence to the skin.

An even more significant factor impacting the effective lifetime of sunscreens is photodegradation of the UV absorbers themselves. Photodegradation refers to a chemical change that occurs upon exposure of a molecule to electromagnetic radiation. While sunscreens containing UV absorbers may be effective for protecting a wearer's skin against the harmful effects of UV radiation, free UV absorbers eventually undergo photodegradation in the course of providing UV protection. Thus, even if a sunscreen formulation remains upon the skin for an extended time period, it eventually becomes necessary to reapply the sunscreen to replenish the UV absorber upon the skin.

The present disclosure addresses the foregoing issues and provides related advantages as well. Namely, the present disclosure incorporates one or more UV absorbers within the pore space of selected metal-organic frameworks having high adsorption capacity for the same. By incorporating a UV absorber within the pore space of metal-organic frameworks according to the disclosure herein, a number of beneficial and unexpected results may be realized. Surprisingly, by incorporating UV absorbers within the pore space of a metal-organic framework, significantly increased resistance of the UV absorber toward photodegradation may be realized. The increased resistance toward photodegradation may facilitate longer time periods between applications of a sunscreen containing metal-organic frameworks loaded with a UV absorber. Further surprisingly, the amount of UV radiation absorbed by the UV absorber may increase when incorporated within the pore space of a metal-organic framework, as measured relative to an equivalent amount of free UV absorber not incorporated within a metal-organic framework. The increased UV absorbance effectively means that the extinction coefficient of the UV absorber increases when incorporated within the pore space of the metal-organic framework. By virtue of the increased UV absorbance, sunscreen formulations may utilize less UV absorber incorporated within a metal-organic framework to realize an equivalent amount of protection against UV radiation.

As a further advantage, metal-organic frameworks themselves may physically shield the UV absorber from direct contact with the skin (e.g., through confinement of the UV absorber within the nanoporosity of the MOF) and provide protection against potentially skin-irritating UV absorbers.

In addition to the foregoing advantages, the metal-organic frameworks disclosed herein are effective for promoting high loadings of UV absorbers therein. For example, loading values of about 20 wt % to about 50 wt %, based upon mass of the metal-organic framework may be realized. Furthermore, the UV absorbers may be effectively retained within the pore space of the metal-organic frameworks when blended in a sunscreen formulation (e.g., a lotion, cream, spray, or the like) and when exposed to various physiological pH conditions. Without being bound by theory or mechanism, the facile retention of the UV absorbers within the metal-organic frameworks is believed to result from strong hydrophobic interactions between the UV absorber and the hydrophobic cavity of the metal-organic framework. When the hydrophobic cavity is of a suitable size and approximately matches the kinetic diameter of the UV absorber, the UV absorber may be stably retained within the pore space of the metal-organic framework. Additional supramolecular interactions may also promote retention stability of the UV absorber within the pore space. The amount of UV absorber incorporated within the pore space of the metal-organic frameworks may be further optimized by the components from which the metal-organic framework is synthesized (e.g., different metal ions and/or counterions), as well as the average particle size of the metal-organic frameworks themselves. Surprisingly, smaller particle sizes may favor greater incorporation of UV absorber therein.

In the present disclosure, suitable metal-organic frameworks may comprise hydrophobic cavities having a pore diameter of about 1 nm or greater, such as about 1 nm to about 2 nm. Such metal-organic frameworks may accommodate UV absorbers in various loading quantities, up to about 50 wt %, as discussed subsequently.

Example metal-organic frameworks that may be suitable for incorporating UV absorbers therein include those such as, for example, ZIF-8, MAF-6, UiO-66-NH₂, MIL-100(Fe), β-cyclodextrin-MOF (β-CD-MOF), hydroxypropyl-β-cyclodextrin-MOF (HP-β-CD-MOF), and γ-cyclodextrin-MOF. Certain metal-organic frameworks may be particularly suitable by virtue of their ability to accommodate high loadings of UV absorbers in amounts ranging from about 20 wt % to about 50 wt %. The loading of the UV absorber may be tailored for each MOF through optimization of the loading technique, sizing of the metal-organic framework, and/or variations in how the metal-organic framework is synthesized (e.g., different metals, counterion variations, temperature variations, and the like). Additional details concerning how UV absorbers become incorporated within the pore space of the various metal-organic frameworks is provided below.

MAF-6, β-CD-MOF (e.g., β-CD-K-MOF), HP-β-CD-MOF (e.g., HP-β-CD-K-MOF), and γ-CD-MOF (e.g., γ-CD-K-MOF) may be particularly advantageous metal-organic frameworks for use as proposed herein, given the known or potential biocompatibility of these materials and high loading capacity values for UV absorbers in amounts up to about 50 wt % in some cases. The above metal-organic frameworks may be advantageous due to loading capacity values of about 20 wt % to about 50 wt %, depending on the particular metal-organic framework and adsorbate employed. β-CD-MOFs contain seven glucose units arranged as a cyclic oligomer, and γ-CD-MOFs contain eight glucose units arranged as a cyclic oligomer, wherein the inner diameter of the cyclic oligomer defines the pore size of the metal-organic framework. β-CD-MOFs, HP-β-CD-MOFs, and γ-CD-MOFs may be conveniently synthesized in water at low pressures, whereas organic solvents under high-temperature and/or high-pressure conditions may be needed for other types of metal-organic frameworks. Advantageously, MAF-6 may be synthesized using alcohol solvents but without using high-pressure synthesis conditions. Raw materials used for synthesizing these types of metal-organic frameworks are not especially expensive.

In some examples, the metal-organic framework may comprise MAF-6. MAF-6 is a reaction product of 2-ethylimidazole and ZnO and has a RHO topology. MAF-6 may accommodate UV absorbers at high loadings. For example, MAF-6 may accommodate the UV-absorber avobenzone in an amount up to about 50 wt %, based on total mass of the metal-organic framework. The structure of MAF-6 includes truncated cuboctahedral supercages and octagonal prisms (cage aperture size=0.76 nm, pore size=2 nm, pore volume=0.61 cm³/g, and BET surface area=1485 m²/g). Each supercage is linked to six neighboring supercages through prisms along the crystallographic axes.

As a demonstration of one aspect of the tunability of metal-organic frameworks, the related metal-organic framework ZIF-8 is formed as a reaction product of 2-methylimidazole and ZnO and instead has a SOD topology. The additional methylene group in the ligand (2-ethylimidazole) of MAF-6 results in a metal-organic framework having higher hydrophobicity within the pore structure and accordingly better accommodation and retention of hydrophobic UV absorbers (e.g., avobenzone) therein, such as through van der Waals and other non-covalent interactions. For example, in addition to having a different topology resulting from the ligand choice, ZIF-8 has a loading capacity for avobenzone that is approximately 10% lower than for MAF-6. In addition to promoting retention of the UV absorber within the pore space of the metal-organic framework, the non-covalent interactions are believed to promote enhanced photostability of the UV absorber, as discussed further hereinbelow.

In some examples, the metal-organic framework may comprise a cyclodextrin-based metal-organic framework. Two cyclodextrin-based metal-organic frameworks that may be suitable include β-CD-MOF (e.g., β-CD-K-MOF) and HP-β-CD-MOF (e.g., HP-β-CD-K-MOF), both of which may accommodate UV absorbers in high amounts. For example, β-CD-K-MOF may accommodate avobenzone in an amount up to about 35 wt % and HP-β-CD-K-MOF may accommodate avobenzone in an amount up to about 20 wt %. Cyclodextrin-based metal-organic frameworks containing β-cyclodextrin may alternately be synthesized using other metal cations (e.g., Na⁺, Mg²⁺, Ca²⁺, Sr⁺², Ba²⁺, Zn²⁺, or combinations thereof) and/or various anions to tailor the amount of UV absorber accommodated therein.

While β-CD-K-MOF is crystalline, HP-β-CD-K-MOF is more semi-crystalline, based on its relatively weak X-ray powder diffraction pattern. The structure of β-CD-K-MOF includes a double channel structure having K⁺ and β-CD molecules in a 1:1 ratio arranged with K—O coordination bonding. More specifically, each β-CD molecule is bonded with a K⁺ through a “T” shaped arrangement to form a hole with a bowl-like assembly, thereby forming a chain structure of two-dimensional (2-D) layers in the crystallographic b-c planes. The “T”-shaped units along the b axis form the double channel structure. Both β-CD-K-MOF and HP-β-CD-K-MOF are able to strongly incorporate UV absorbers through their hydrophobic cavities.

Another example of a suitable cyclodextrin-based metal organic framework is γ-CD-MOF. γ-CD-K-MOF may accommodate avobenzone in an amount up to about 20 wt %.

The particle size of the metal-organic frameworks may vary to some degree depending on the components used during their synthesis. Following synthesis, the particle size of the metal-organic frameworks may be further decreased, if desired, such as through milling, sonication, or similar techniques. Surprisingly, smaller particle sizes for the metal-organic frameworks may accommodate higher amounts of UV absorbers or other adsorbates. Further, smaller particle sizes of the metal-organic frameworks may afford increased stability of the UV absorbers toward UV radiation.

The average particle size of the metal-organic frameworks may be selected to promote incorporation of a desired amount of UV absorber or other adsorbate therein. In non-limiting examples, the metal-organic frameworks, prior to incorporation of a UV absorber or other adsorbate therein may have an average particle size of about 50 nm to about 2 microns, or about 60 nm to about 1 micron, or about 100 nm to about 500 nm, or about 200 nm to about 400 nm, or about 125 nm to about 150 nm, or about 250 nm to about 350 nm. The average particle size may be measured by dynamic light scattering or by measurement of individual particles in an SEM image.

The metal-organic frameworks may have a BET surface area of about 1000 m²/g to about 2000 m²/g, such as about 1485 m²/g.

The metal-organic frameworks may have an adsorption capacity of up to about 50 wt %, based on mass of the metal-organic framework, or up to about 40 wt %, based on mass of the metal-organic framework, or up to about 30 wt %, based on mass of the metal-organic framework, or up to about 25 wt %, based on mass of the metal-organic framework. A suitable minimum absorption capacity may be about 20 wt %, based on mass of the metal-organic framework.

Any UV absorber or other adsorbate that may become successfully incorporated within a given metal-organic frameworks disclosed herein may be utilized in the compositions of the present disclosure. Non-limiting examples of UV absorbers that may be utilized include, for example, avobenzone (AVB), octyl p-methoxycinnamate (OMC), or any combination thereof. Other UV absorbers suitable for use in sunscreen or other personal care products may also be included.

Other suitable adsorbates that may be present within the metal-organic frameworks include, for example, at least one retinol compound. Suitable retinol compounds may include, for example, retinol, retinoic acid, retinoic acid esters, retinaldehyde, and the like.

Still other examples of suitable adsorbates that may be present within the metal-organic frameworks include, for instance, an acid, a vitamin, a pro-vitamin, a humectant, an antibacterial agent, an antifungal agent, an antiviral agent, an antiparasitic agent, an antipruritic agent, an analgesic agent, an anesthetic agent, a nonsteroidal anti-inflammatory drug, a keratolytic agent, an alpha hydroxy acid, an beta hydroxy acid, ascorbic acid, niacinamide, panthenol, vitamin C, vitamin B, triethylene glycol, tripropylene glycol, propylene glycol, polypropylene glycol, glycerin, sorbitol, hexylene glycol, butylene glycol, urea, a sulfonamide, tetracycline hydrochloride, triclosan, bacitracin, polymyxin B, neomycin, gentamycin, meclocycline, sulfacetamide sodium, an imidazole, a triazole, a tetrazole, benzoyl peroxide, allantoin, sulfur, a mupirocin, erythromycin, clindamycin, acyclovir, penciclovir, docosanol, selenium disulfide, zinc pyrithione, permethrin, lindane, crotamiton, oxybenzone, octisalate, octocrylene, homosalate, octinoxate, retinol, retinol palmitate, tocopherol, tocotrienol, zinc pyrithione, benzocaine, butamben, dibucaine, lidocaine, tetracaine, pramocaine, proxymetacaine, prilocaine, diclofenac, fentanyl, capsaicin, a salicylate, an antihistamine, a corticosteroid, ibuprofen, naproxen, and the like. In some instances, by confining an odiferous adsorbate within a metal-organic framework, the odor may decrease relative to that of the free adsorbate. Likewise, in some instances, a fragrant adsorbate may be incorporated with the metal-organic framework, and slow release of the fragrant adsorbate may convey a pleasing smell of the compositions disclosed herein. In some instances, heat-labile adsorbates may be incorporated within the metal-organic frameworks and exhibit higher thermal stability than do the corresponding free adsorbates.

The metal-organic frameworks disclosed herein may include one or more UV absorbers incorporated within the pore space therein. For example, the metal-organic frameworks may incorporate avobenzone alone within the pore space therein, octyl p-methoxycinnamate alone within the pore space therein, or a combination of avobenzone and octyl p-methoxycinnamate within the pore space therein. When multiple UV absorbers are incorporated within a given metal-organic framework, the amount of each UV adsorber that is incorporated may be substantially the same or may differ depending on the metal-organic framework and the affinity for each UV absorber for the pore space therein. For instance, competitive binding for limited pore space may lead to incorporation of more of a first adsorbate compared to a second adsorbate. As a non-limiting example, β-CD-K-MOF may be particularly effective for concurrently incorporating both avobenzone and octyl p-methoxycinnamate in roughly equal amounts, whereas other metal-organic frameworks may incorporate one UV absorber in preference to the other. Other metal-organic frameworks may incorporate avobenzone in preference to octyl p-methoxycinnamate, though both UV absorbers may be incorporated to at least some degree in combination with each other in most cases.

When incorporated within the pore space of the metal-organic framework, the UV absorber may undergo less photodegradation compared to the free UV absorber (UV absorber not incorporated with a metal-organic framework). The amount of photodegradation may be determined by irradiation with UV-A radiation (365 nm wavelength at 100 W) for a period of time (4 hr), followed by analysis of the remaining UV absorber using UV-VIS spectroscopy. For example, avobenzone incorporated within a metal-organic framework may exhibit about 35% or less photodegradation, when subjected to 365 nm radiation for up to 8 hours. The amount of photodegradation may be determined by UV-VIS spectroscopy and measured relative to the UV-VIS absorbance of an equivalent amount of free avobenzone not incorporated within a metal-organic framework. The amount of photodegradation of avobenzone may be less than 25% over 5 hours of UV-A radiation, or less than 15% photodegradation over 2 hours of UV radiation.

Similarly, octyl p-methoxycinnamate may exhibit about 25% or less photodegradation up to 4 hours, and about 15% or less photodegradation up to 1 hour, whereas free octyl p-methoxycinnamate exhibits considerably higher photodegradation over the same time periods. For example, free octyl p-methoxycinnamate exhibits greater than 30% photodegradation when irradiated for 1 hour under similar conditions.

When incorporated within a metal-organic framework, the UV absorber may exhibit increased UV absorption at λₘₐₓ, as compared to an equivalent concentration of the free UV absorber. The term “λₘₐₓ” refers to the wavelength at which the UV absorption is greatest. Although increased UV absorption may be realized at λₘₐₓ, it is to be appreciated that the UV absorption may also increase at other absorption wavelengths as well. In non-limiting examples, the UV absorber incorporated within the metal-organic framework may exhibit up to about 25% increased absorbance at λₘₐₓ or up to about 10% increased absorbance at λₘₐₓ, each as compared to an equivalent concentration of free UV absorber.

The UV absorber incorporated within the metal-organic framework may be relatively stable to a range of pH conditions, such as about 5.5, about 6.3, or about 7.4, corresponding to the pH values of sweat, normal physiological pH, and blood. Stability may be assessed by measuring the UV-VIS absorbance of the supernatant liquid obtained following contacting the metal-organic framework with an aqueous buffer having a specified pH value. The amount of UV absorber released at a pH value of 5.5 may be less than about 1 wt %, and the amount of UV absorber released at a pH value of 6.3 or 7.4 may be less than about 0.7 wt %.

The manner in which the UV absorber or other adsorbate becomes incorporated within the pore space of the metal-organic frameworks is not believed to be particularly limited. Depending on the metal-organic framework being loaded with adsorbate, loading may take place by grinding the adsorbate and the metal-organic framework together (mechanochemical loading, preferably in the absence of solvent), heating the adsorbate together with the metal-organic framework in a solvent (solvent-assisted loading), or any combination thereof. In some cases, the adsorbate may be incorporated within the pore space of the metal-organic framework by synthesizing the metal-organic framework in the presence of the adsorbate. MAF-6 may be synthesized in the latter manner.

In the case of MAF-6, solvent-assisted loading of adsorbates, such as avobenzone and octyl p-methoxycinnamate, may be effective for filling the pore space of the metal-organic frameworks. For example, the adsorbate and the metal-organic framework may be heated in methanol solvent at about 70-80° C. In contrast, mechanochemical loading may be more effective for loading adsorbates within cyclodextrin-based metal-organic frameworks, such as β-CD-K-MOF, HP-β-CD-K-MOF, and γ-CD-K-MOF.

The metal-organic frameworks loaded with a UV absorber or other hydrophobic adsorbate according to the disclosure herein may be utilized in various further compositions, which may be a personal care product. Personal care products may include formulations such as sunscreens, cosmetics, foundations, and the like. Conventional personal care product formulations for creams, gels, lotions, foundation, and the like may be utilized to disperse the metal-organic frameworks loaded with UV absorber with other components.

The personal care products disclosed herein may be in any liquid, semi-solid, or solid form, wherein suitable liquid forms may be an emulsion, solution, or suspension, such as an aqueous emulsion or solution. Suitable liquid forms may comprise about 5 wt % to about 99.9 wt % water, based on total mass. Solid forms may include, for example, sticks, powders, and the like.

Physiologically acceptable components that may be incorporated within the personal care products may be selected by one having ordinary skill in the art. Examples of physiologically acceptable components that may be utilized in personal care products along with the metal-organic frameworks include adjuvants suitable for a given formulation, such as solvents, thickeners, diluents, antioxidants, colorants, other UV absorbers and sun filters, self-tanning agents, pigments, fillers, preservatives, perfumes, odor absorbers, essential oils, vitamins, essential fatty acids, surfactants, film-forming polymers, and the like. Thus, in some instances a UV absorber incorporated within the pore space of a metal-organic framework may be formulated with a UV absorber that is not incorporated within the porosity of the metal-organic framework. The UV absorber not incorporated within the pore space of the metal-organic framework may be the same as or different than the UV absorber incorporated within the porosity of the metal-organic framework. In some cases, the UV absorber not incorporated within the pores space of the metal-organic framework may be incapable of entering the pore space of the metal-organic framework.

Additional active agents may also be present within personal care products containing the metal-organic frameworks. Suitable active agents that may be optionally present include, but are not limited to, substances having properties such as, anti-aging, firming, lightening, moisturizing, draining, microcirculation promoting, exfoliating, desquamating, extracellular matrix stimulating, energy metabolism activating, antibacterial, antifungal, soothing, anti-free radical, anti-UV, anti-acne, anti-inflammatory, anesthetic, warm feeling-inducing, cool feeling-inducing, slimming, the like, and any combination thereof.

Additional components that may be present in personal care product formulations include, for example, preservatives, antioxidants, pH adjusters, solvents, oils, humectants, surfactants, vitamins, antioxidants, fragrances, minerals, particulates, and inert ingredients such as silicones. Examples of such additional components will likewise be familiar to persons having ordinary skill in the art of formulating personal care products.

The personal care products disclosed herein may be applied topically to the skin as part of a user's skincare routine, particularly when protection from the sun is needed. The personal care products may be applied to the skin in a desired location one or more times a day on an as-needed basis.

Embodiments disclosed herein include the following:
Embodiment 1. A metal-organic framework comprising hydrophobic cavities having a pore diameter of about 1 nm to about 2 nm, and, optionally, at least one adsorbate within the hydrophobic cavities.
Embodiment 2. The metal-organic framework of Embodiment 1, wherein the hydrophobic cavities have an aperture size of about 0.76 nm.
Embodiment 3. The metal-organic framework of Embodiment 1 or Embodiment 2, wherein the metal-organic framework comprises a zeolitic imidazolate having RHO topology and cuboctahedral hydrophobic cavities of about 2 nm pore diameter.
Embodiment 4. The metal-organic framework of Embodiment 3, wherein the metal-organic framework has an average particle size of about 50 nm to about 2 microns.
Embodiment 5. The metal-organic framework of Embodiment 3, wherein the metal-organic framework has an average particle size of about 60 nm to about 1 micron.
Embodiment 6. The metal-organic framework of Embodiment 3, wherein the metal-organic framework has an average particle size of about 250 nm to about 350 nm.
Embodiment 7. The metal-organic framework of Embodiment 3, wherein the metal-organic framework has an average particle size of about 125 nm to about 150 nm.
Embodiment 8. The metal-organic framework of any one of Embodiments 3-7, wherein the metal-organic framework has a BET surface area of about 1000 m²/gm to about 2000 m²/gm.
Embodiment 9. The metal-organic framework of any one of Embodiments 3-8, wherein the metal-organic framework has a BET surface area of about 1485 m²/gm.
Embodiment 10. The metal-organic framework of any one of Embodiments 3-9, wherein the metal-organic framework has an adsorption capacity of up to about 50 wt %, based on mass of the metal-organic framework.
Embodiment 11. The metal-organic framework of any one of Embodiments 3-9, wherein the metal-organic framework has an adsorption capacity of up to about 25 wt %, based on mass of the metal-organic framework.
Embodiment 12. The metal-organic framework of any one of Embodiments 3-10, wherein the at least one adsorbate is present.
Embodiment 13. The metal-organic framework of Embodiment 12, wherein the at least one adsorbate comprises at least one hydrophobic adsorbate.
Embodiment 14. The metal-organic framework of Embodiment 13, wherein the at least one hydrophobic adsorbate comprises at least one retinol adsorbate.
Embodiment 15. The metal-organic framework of Embodiment 13, wherein the at least one hydrophobic adsorbate comprises at least one of avobenzone and octyl p-methoxycinnamate.
Embodiment 16. The metal-organic framework of Embodiment 15, wherein the avobenzone, when incorporated within the metal-organic framework, exhibits less than 35% photodegradation when subjected to UV-A radiation for up to 8 hours.
Embodiment 17. The metal-organic framework of Embodiment 15, wherein the avobenzone, when incorporated within the metal-organic framework, exhibits less than 30% photodegradation when subjected to UV-A radiation for up to 4 hours.
Embodiment 18. The metal-organic framework of Embodiment 15, wherein the avobenzone, when incorporated within the metal-organic framework, exhibits less than 25% photodegradation when subjected to UV-A radiation for up to 5 hours.
Embodiment 19. The metal-organic framework of Embodiment 15, wherein the avobenzone, when incorporated within the metal-organic framework, exhibits less than 15% photodegradation when subjected to UV-A radiation for up to 2 hours.
Embodiment 20. The metal-organic framework of Embodiment 15, wherein the octyl p-methoxycinnamate, when incorporated within the metal-organic framework, exhibits less than 25% photodegradation when subjected to UV-A radiation for up to 4 hours.
Embodiment 21. The metal-organic framework of Embodiment 15, wherein the octyl p-methoxycinnamate, when incorporated within the metal-organic framework, exhibits less than 15% photodegradation when subjected to UV-A radiation for up to 1 hour.
Embodiment 22. The metal-organic framework of any one of Embodiments 12-21, wherein the hydrophobic adsorbate, when incorporated within the metal-organic framework, exhibits up to about 25% increased absorbance at λₘₐₓ as compared to an equivalent concentration of free hydrophobic adsorbate.
Embodiment 23. The metal-organic framework of any one of Embodiments 12-21, wherein the hydrophobic adsorbate, when incorporated within the metal-organic framework, exhibits up to about 10% increased absorbance at λₘₐₓ as compared to an equivalent concentration of free hydrophobic adsorbate.
Embodiment 24. A composition comprising:
a metal-organic framework having a plurality of pores defined therein; and
at least one hydrophobic adsorbate incorporated within the plurality of pores;
   wherein the at least one hydrophobic adsorbate is present within the plurality of pores in an amount ranging from about 20 wt % to about 50 wt %, based upon mass of the metal-organic framework; and
   wherein photodegradation of the hydrophobic adsorbate, when incorporated within the plurality of pores, is at least partially suppressed relative to free hydrophobic adsorbate not incorporated within the plurality of pores.
Embodiment 25. The composition of Embodiment 24, wherein the pores have a pore diameter of about 1 nm to about 2 nm.
Embodiment 26. The composition of Embodiment 24, wherein the metal-organic framework is MAF-6.
Embodiment 27. The composition of Embodiment 24, wherein the metal-organic framework is ZIF-8
Embodiment 28. The composition of Embodiment 24, wherein the metal-organic framework is MIL-100(Fe).
Embodiment 29. The composition of Embodiment 24, wherein the metal-organic framework is UiO-66-NH₂.
Embodiment 30. The composition of any one of Embodiments 24-29, wherein the at least one hydrophobic adsorbate comprises a UV-absorber.
Embodiment 31. The composition of Embodiment 30, wherein the UV-absorber comprises avobenzone, octyl p-methoxycinnamate, or any combination thereof.
Embodiment 32. The composition of Embodiment 30, wherein the UV-absorber comprises at least one retinol compound.
Embodiment 33. The composition of Embodiment 31, wherein the metal-organic framework is MAF-6.
Embodiment 34. The composition of Embodiment 33, wherein the MAF-6 has an average particle size of about 125 nm to about 350 nm.
Embodiment 35. A personal care composition comprising the composition of any one of Embodiments 24-35 or any one of Embodiments 1-23.

Additional embodiments disclosed herein include the following:
Embodiment 1A. A cyclodextrin-based metal-organic framework comprising a cyclodextrin defining a plurality of pores having a pore size of at least about 1 nm in diameter, and, optionally, at least one adsorbate within the plurality of pores.
Embodiment 2A. The cyclodextrin-based metal-organic framework of Embodiment 1A, wherein the cyclodextrin comprises a β-cyclodextrin.
Embodiment 3A. The cyclodextrin-based metal-organic framework of Embodiment 2A, wherein the β-cyclodextrin comprises at least one metal ion selected from the group consisting of K⁺, Na⁺, Mg²⁺, Ca²⁺, Sr⁺², Ba²⁺, Zn²⁺, and any combination thereof.
Embodiment 4A. The cyclodextrin-based metal-organic framework of Embodiment 3A, wherein the at least one metal ion comprises K⁺.
Embodiment 5A. The cyclodextrin-based metal-organic framework of Embodiment 3A or Embodiment 4A, wherein the cyclodextrin-based metal-organic framework has an average particle size of about 50 nm to about 2 microns.
Embodiment 6A. The cyclodextrin-based metal-organic framework of Embodiment 3A or Embodiment 4A, wherein the cyclodextrin-based metal-organic framework has an average particle size of about 60 nm to about 1 micron.
Embodiment 7A. The cyclodextrin-based metal-organic framework of Embodiment 3A or Embodiment 4A, wherein the cyclodextrin-based metal-organic framework has an average particle size of about 250 nm to about 350 nm.
Embodiment 8A. The cyclodextrin-based metal-organic framework of any one of Embodiments 3A-7A, wherein the cyclodextrin-based metal-organic framework has an adsorption capacity of up to about 35 wt %, based on mass of the metal-organic framework.
Embodiment 9A. The cyclodextrin-based metal-organic framework of any one of Embodiments 3A-7A, wherein the cyclodextrin-based metal-organic framework has an adsorption capacity of up to about 25 wt %, based on mass of the metal-organic framework.
Embodiment 10A. The cyclodextrin-based metal-organic framework of any one of Embodiments 3A-9A, wherein the at least one adsorbate is present.
Embodiment 11A. The cyclodextrin-based metal-organic framework of Embodiment 10A, wherein the at least one adsorbate comprises at least one hydrophobic adsorbate.
Embodiment 12A. The cyclodextrin-based metal-organic framework of Embodiment 11A, wherein the at least one hydrophobic adsorbate comprises at least one retinol adsorbate.
Embodiment 13A. The cyclodextrin-based metal-organic framework of Embodiment 11A, wherein the at least one adsorbate comprises at least one of avobenzone and octyl p-methoxycinnamate.
Embodiment 14A. The cyclodextrin-based metal-organic framework of Embodiment 13A, wherein the avobenzone, when incorporated within the cyclodextrin-based metal-organic framework, exhibits less than 35% photodegradation when subjected to UV-A radiation for 4 hours.
Embodiment 15A. The cyclodextrin-based metal-organic framework of Embodiment 13A, wherein the avobenzone, when incorporated within the cyclodextrin-based metal-organic framework, exhibits less than 30% photodegradation when subjected to UV-A radiation for 4 hours.
Embodiment 16A. The cyclodextrin-based metal-organic framework of Embodiment 13A, wherein the avobenzone, when incorporated within the cyclodextrin-based metal-organic framework, exhibits less than 25% photodegradation when subjected to UV-A radiation over 4 hours.
Embodiment 17A. The cyclodextrin-based metal-organic framework of Embodiment 13A, wherein the avobenzone, when incorporated within the cyclodextrin-based metal-organic framework, exhibits less than 20% photodegradation when subjected to UV-A radiation for up to 4 hours.
Embodiment 18A. The cyclodextrin-based metal-organic framework of Embodiment 13A, wherein the avobenzone, when incorporated within the cyclodextrin-based metal-organic framework, exhibits less than 15% photodegradation when subjected to UV-A radiation for up to 4 hours.
Embodiment 19A. The cyclodextrin-based metal-organic framework of Embodiment 13A, wherein the octyl p-methoxycinnamate, when incorporated within the cyclodextrin-based metal-organic framework, exhibits less than 25% photodegradation when subjected to UV-A radiation for up to 5 hours.
Embodiment 20A. The cyclodextrin-based metal-organic framework of Embodiment 13A, wherein the octyl p-methoxycinnamate, when incorporated within the cyclodextrin-based metal-organic framework, exhibits less than 20% photodegradation when subjected to UV-A radiation for up to 5 hours.
Embodiment 21A. The cyclodextrin-based metal-organic framework of Embodiment 13A, wherein the avobenzone, when incorporated within the cyclodextrin-based metal-organic framework, exhibits up to about 40% increased UV-A absorbance, as compared to an equivalent concentration of free avobenzone.
Embodiment 22A. The cyclodextrin-based metal-organic framework of Embodiment 13A, wherein the avobenzone, when incorporated within the cyclodextrin-based metal-organic framework, exhibits up to about 30% increased UV-A absorbance, as compared to an equivalent concentration of free avobenzone.
Embodiment 23A. The cyclodextrin-based metal-organic framework of Embodiment 13A, wherein the avobenzone, when incorporated within the cyclodextrin-based metal-organic framework, exhibits up to about 20% increased UV-A absorbance, as compared to an equivalent concentration of free avobenzone.
Embodiment 24A. The cyclodextrin-based metal-organic framework of Embodiment 13A, wherein the avobenzone, when incorporated within the cyclodextrin-based metal-organic framework, exhibits up to about 10% increased UV-A absorbance, as compared to an equivalent concentration of free avobenzone.
Embodiment 25A. The cyclodextrin-based metal-organic framework of Embodiment 2A, wherein the β-cyclodextrin comprises 2-hydroxypropyl-β-cyclodextrin, and the cyclodextrin-based metal-organic framework comprises K⁺.
Embodiment 26A. The cyclodextrin-based metal-organic framework of Embodiment 25A, wherein the cyclodextrin-based metal-organic framework has an average particle size of about 60 nm to about 1 micron.
Embodiment 27A. The cyclodextrin-based metal-organic framework of Embodiment 25A, wherein the cyclodextrin-based metal-organic framework has an average particle size of about 500 nm to about 800 nm.
Embodiment 28A. The cyclodextrin-based metal-organic framework of any one of Embodiments 25A-27A, wherein the at least one adsorbate is present.
Embodiment 29A. The cyclodextrin-based metal-organic framework of Embodiment 28A, wherein the at least one adsorbate comprises at least one hydrophobic adsorbate.
Embodiment 30A. The cyclodextrin-based metal-organic framework of Embodiment 29A, wherein the at least one hydrophobic adsorbate comprises at least one retinol adsorbate.
Embodiment 31A. The cyclodextrin-based metal-organic framework of Embodiment 29A, wherein the at least one hydrophobic adsorbate comprises at least one of avobenzone and octyl p-methoxycinnamate.
Embodiment 32A. The cyclodextrin-based metal-organic framework of Embodiment 31A, wherein the avobenzone, when incorporated within the cyclodextrin-based metal-organic framework, exhibits less than 60% photodegradation when subjected to UV-A radiation for up to 4 hours.
Embodiment 33A. The cyclodextrin-based metal-organic framework of Embodiment 31A, wherein the avobenzone, when incorporated within the cyclodextrin-based metal-organic framework, exhibits less than 50% photodegradation when subjected to UV-A radiation for up to 4 hours.
Embodiment 34A. The cyclodextrin-based metal-organic framework of Embodiment 31A, wherein the cyclodextrin-based metal-organic framework has an adsorption capacity of up to about 30 wt %, based on mass of the metal-organic framework.
Embodiment 35A. The cyclodextrin-based metal-organic framework of Embodiment 31A, wherein the cyclodextrin-based metal-organic framework has an adsorption capacity of up to about 20 wt %, based on mass of the metal-organic framework.
Embodiment 36A. The cyclodextrin-based metal-organic framework of Embodiment 1A, wherein the cyclodextrin comprises γ-cyclodextrin.
Embodiment 37A. The cyclodextrin-based metal-organic framework of Embodiment 36, wherein the γ-cyclodextrin comprises at least one metal ion selected from the group consisting of K⁺, Na⁺, Mg²⁺, Ca²⁺, Sr⁺², Ba²⁺, Zn²⁺, and any combination thereof.
Embodiment 38A. The cyclodextrin-based metal-organic framework of Embodiment 37A, wherein the at least one metal ion comprises K⁺.
Embodiment 39A. The cyclodextrin-based metal-organic framework of Embodiment 37A or Embodiment 38A, wherein the at least one adsorbate is present.
Embodiment 40A. The cyclodextrin-based metal-organic framework of Embodiment 39A, wherein the at least one adsorbate comprises at least one hydrophobic adsorbate.
Embodiment 41A. The cyclodextrin-based metal-organic framework of Embodiment 40A, wherein the at least one hydrophobic adsorbate comprises at least one retinol adsorbate.
Embodiment 42A. The cyclodextrin-based metal-organic framework of Embodiment 40A, wherein the at least one hydrophobic adsorbate comprises at least one of avobenzone and octyl p-methoxycinnamate.
Embodiment 43A. A composition comprising:
a cyclodextrin-based metal-organic framework comprising a cyclodextrin defining a plurality of pores therein; and
at least one hydrophobic adsorbate incorporated within the plurality of pores;
   wherein the at least one hydrophobic adsorbate is present within the plurality of pores in an amount ranging from about 20 wt % to about 35 wt %, based upon mass of the metal-organic framework; and
   wherein photodegradation of the hydrophobic adsorbate, when incorporated within the plurality of pores, is at least partially suppressed relative to free hydrophobic adsorbate not incorporated within the plurality of pores.
Embodiment 44A. The composition of Embodiment 43A, wherein the cyclodextrin is a β-cyclodextrin.
Embodiment 45A. The composition of Embodiment 44A, wherein the β-cyclodextrin is hydroxypropyl-β-cyclodextrin.
Embodiment 46A. The composition of Embodiment 44A or Embodiment 45A, wherein the at least one hydrophobic adsorbate comprises a UV-absorber.
Embodiment 47A. The composition of Embodiment 46A, wherein the UV-absorber comprises avobenzone, octyl p-methoxycinnamate, or any combination thereof.
Embodiment 48A. The composition of Embodiment 46A, wherein the UV-absorber comprises at least one retinol compound.
Embodiment 49A. The composition of Embodiment 43A, wherein the cyclodextrin is a γ-cyclodextrin.
Embodiment 50A. The composition of Embodiment 49A, wherein the at least one hydrophobic adsorbate comprises a UV-absorber.
Embodiment 51A. The composition of Embodiment 50A, wherein the UV-absorber comprises avobenzone, octyl p-methoxycinnamate, or any combination thereof.
Embodiment 52A. The composition of Embodiment 50A, wherein the UV-absorber comprises at least one retinol compound.
Embodiment 53A. A personal care product comprising the composition of any one of Embodiments 43A-52A.

To facilitate a better understanding of the present disclosure, the following examples of preferred or representative embodiments are given. In no way should the following examples be read to limit, or to define, the scope of the invention.

### EXAMPLES

The following metal-organic frameworks (MOFs) were assessed for their ability to uptake various adsorbates (guest molecules): ZIF-8, MAF-6, UiO-66-NH₂, UiO-67, MIL-100(Fe), β-cyclodextrin-K-MOF (β-CD-K-MOF), γ-cyclodextrin-MOF (γ-CD-MOF), and hydroxypropyl β-cyclodextrin-K-MOF (HP-β-CD-K-MOF). As appropriate, the various MOFs were either synthesized according to literature procedures or as outlined below. MAF-6, β-CD-K-MOF, and HP-β-CD-K-MOF were selected for further study based on their high uptake of avobenzone. Procedures for incorporating avobenzone and other adsorbates in the various MOFs are outlined below. In the description below, the “@” symbol indicates that an adsorbate is incorporated within the pore space of the metal-organic framework.

### Example 1: Loading of MAF-6 with Adsorbates and Characterization Thereof

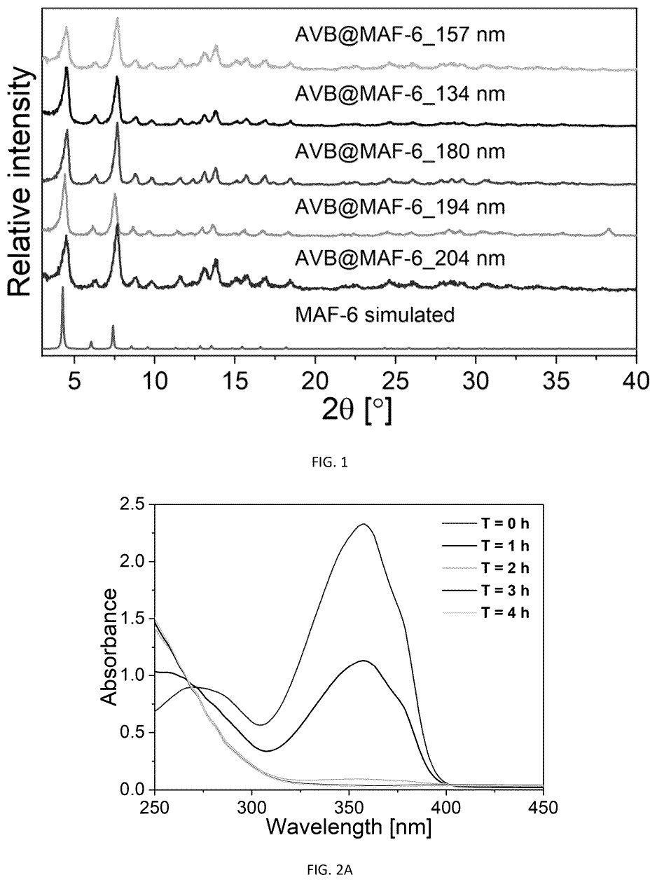

A. Synthesis of MAF-6 by Milling. A mixture of ZnO (81.38 mg, 1 mmol), 2-ethylimidazole (385 mg, 4 mmol) and ethanol (200 ml) were milled using a ball milling machine for 1 hour. The resulting white solid was washed three times with methanol, centrifuged, air dried and finally oven dried at 80° C.
B. Loading of Avobenzone in Previously Synthesized MAF-6 (AVB@MAF-6). 1 g of avobenzone was dissolved in 200 mL of MeOH. To this solution was added 1 g of MAF-6, and the reaction mixture was heated at 70° C. overnight. The reaction mixture was cooled to room temperature and filtered. The residue solid was washed with methanol, air dried, and finally dried in an oven at 80° C. for 12 h. The loading of avobenzone within the MAF-6 exceeded 50 wt %, based on mass of the original MAF-6.
C: Synthesis of Maf-6 and in Situ Loading With Avobenzone (avb@maf-6). MAF-6 was synthesized and loaded in situ with avobenzone in an Attritor Mill by combining 1.62 g ZnO, 5.76 g 2-ethylimidazole, 1 g avobenzone, and 4 mL ethanol and milling the combined mixture for 1 hour. Particles of AVB@MAF-6 having an average particle size ranging from 134 nm to 204 nm were obtained, depending on the milling conditions. FIG. 1 is an X-Ray powder diffraction pattern of various particle sizes of AVB@MAF-6. As shown, the same crystalline phase was obtained in each case. The X-Ray powder diffraction patterns were similar to that obtained when avobenzone was incorporated in previously synthesized MAF-6 (not shown).
D: Synthesis of MAF-6 with Nanoscale Particle Sizes. 2-Ethylimidazole and ZnO were combined at a 3:1 mass ratio in cyclohexane and isopropanol in the presence of 25% aqueous ammonia and varying concentrations of surfactants. MOF particles were then allowed to grow by slow nucleation. In the presence of 0.03 wt % cethyltrimethylammonium bromide (CTAB), the average particle size was 264±10 nm, and in the presence of 0.05 wt % CTAB, the average particle size was 240±11 nm. Replacement of the 0.05 wt % CTAB with 0.05 wt % polyvinylpyrollidone (PVP) lowered the average particle size to 217±12 nm.

Loading with avobenzone was then conducted as above. The avobenzone loading was approximately 50 wt %, based upon the weight of the original metal-organic framework.
E: Solvent-Assisted Loading of MAF-6 with Octyl p-methoxycinnamate (OMC@MAF-6). Previously synthesized MAF-6 and octyl p-methoxycinnamate (OMC) were combined at a 1:2 mass ratio and heated neat at 70° C. in the absence of solvent. The loading of octyl p-methoxycinnamate within the MOF was 21.66 wt %, based upon total mass of the MAF-6, as determined by UV-VIS spectroscopy.
F: Solvent-Assisted Loading of MAF-6 with Avobenzone and Octyl p-methoxycinnamate (AVB/OMC@MAF-6). Previously synthesized MAF-6, avobenzone, and octyl p-methoxycinnamate (OMC) were combined at a 1:2:2 mass ratio and heated neat at 70° C. in the absence of solvent. No OMC was incorporated within the MOF. The loading of avobenzone within the MOF was 50.6 wt %, based upon total mass of the MAF-6, as determined by UV-VIS spectroscopy.
G: Photostability of AVB@MAF-6 Dispersed in Methanol. Free avobenzone and AVB@MAF-6 were dispersed in methanol at equivalent loadings of avobenzone, and the samples were irradiated at 365 nm UV radiation (UV-A radiation) using a 100 W lamp located 14 cm from the sample. UV-VIS spectra were taken for both samples during the irradiation time period. FIG. 2A shows overlaid UV-VIS spectra of free avobenzone dissolved in methanol and irradiated for various times at 365 nm using a 100 W lamp located 14 cm from the sample. As shown, there was a sharp decrease in the avobenzone absorbance peak, and essentially full photodegradation was reached at 2 hours (˜50% photodegradation at 1 hour). FIG. 2B shows overlaid UV-VIS spectra of AVB@MAF-6 (average particle size=564 nm) dispersed in methanol and irradiated for various times at 365 nm using a 100 W lamp located 14 cm from the sample. Unlike free avobenzone, the photodegradation of avobenzone was significantly decreased when incorporated within the MOF. As shown, there was only approximately 2% photodegradation at 2 hours of irradiation, and at the maximum testing time (9 hours), the amount of photodegradation had risen to only approximately 18%, as measured by the decrease in UV absorbance at λₘₐₓ. Slightly increased avobenzone absorbance occurred at wavelengths between 250 nm and 300 nm.

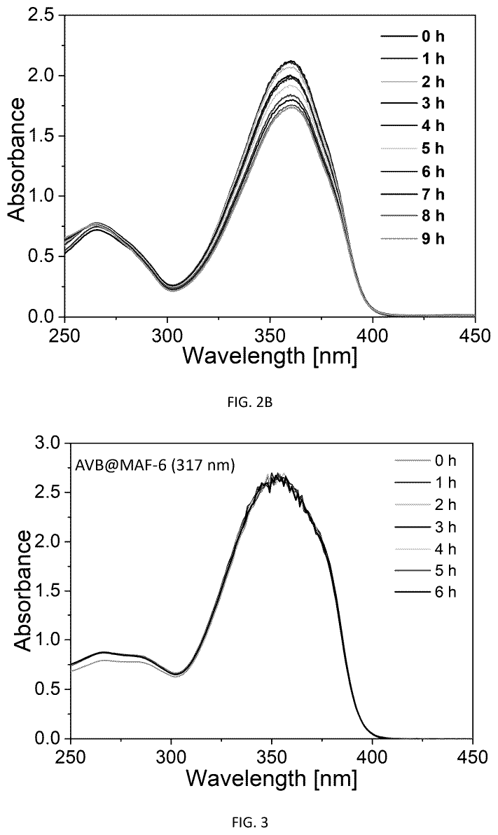

Photostability of avobenzone was also assessed in AVB@MAF-6 having smaller particle sizes (see D above). Like the previous measurements, the AVB@MAF-6 was dispersed in methanol while being irradiated with the UV-A radiation. Surprisingly, increased photostability of avobenzone was realized at the smaller particle sizes. FIG. 3 shows overlaid UV-VIS spectra of AVB@MAF-6 dispersed in methanol and having an average particle size of 317 nm irradiated for various times at 365 nm using a 100 W lamp located 14 cm from the sample. In comparison to AVB@MAF-6 having larger particle sizes (see FIG. 2B, for example), a considerably lower degree of avobenzone photodegradation was observed.

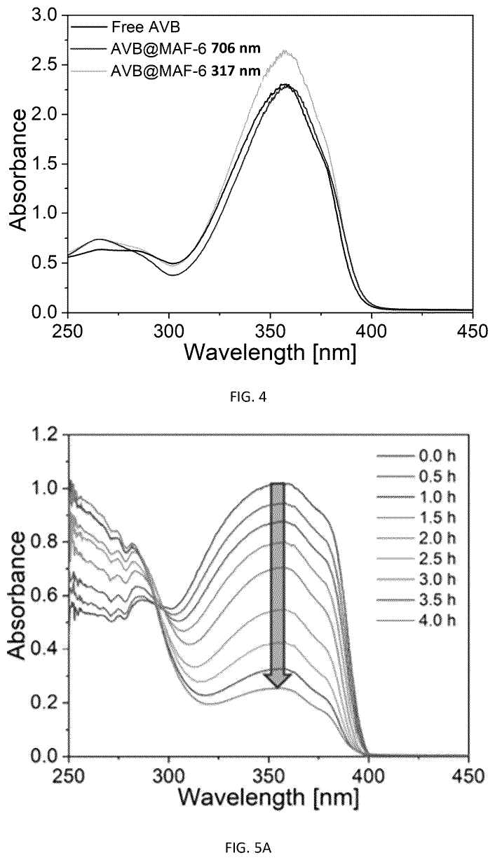

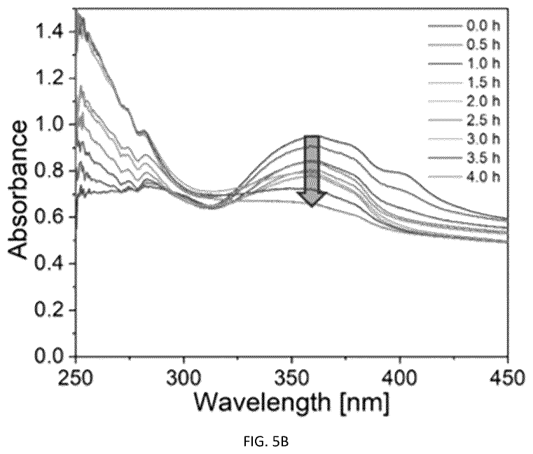

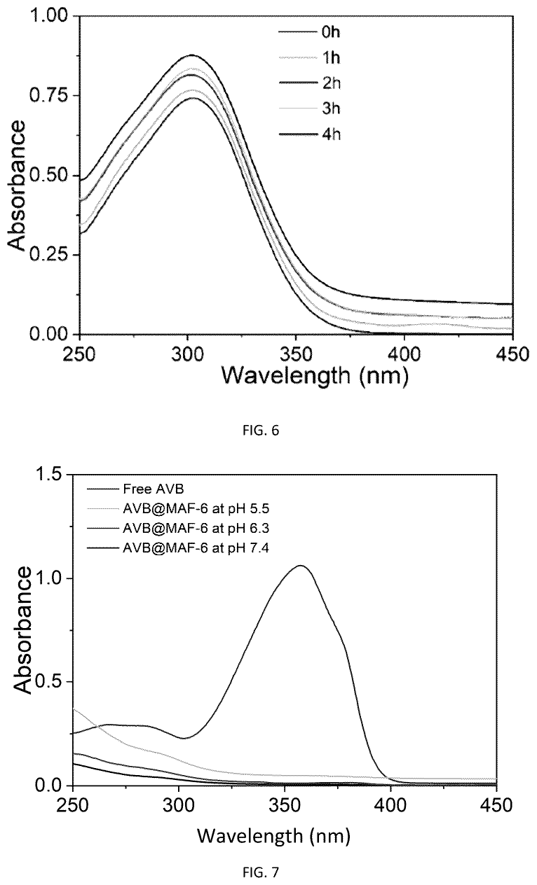

In addition, at smaller AVB@MAF-6 particle sizes, increased UV absorbance for avobenzone was realized relative to an equivalent concentration of avobenzone, either as free avobenzone molecules or as avobenzone incorporated within larger AVB@MAF-6 particles. Like the previous measurements, the AVB@MAF-6 was dispersed in methanol while being irradiated with the UV-A radiation. FIG. 4 shows overlaid UV-VIS spectra of AVB@MAF-6 dispersed in methanol and having various average particle sizes in comparison to free avobenzone. As shown in FIG. 4, free avobenzone and AVB@MAF-6 having an average particle size of 706 nm exhibited approximately the same UV absorbance. In contrast, at an average particle size of 317 nm, a significant increase in UV absorbance (>10%) was realized.
H: Photostability of AVB@MAF-6 as a Thin-Film. Avobenzone and AVB@MAF-6 were separately formulated in a lotion base, such that the avobenzone concentration in the lotion base was 3 wt %, based on total mass. 32.5 mg of each formulation was spread uniformly upon a PMMA plate to provide a thin-film. The thin-films were then irradiated for various times at 365 nm using a 100 W lamp located 14 cm from the sample. UV-VIS spectra were acquired every 0.5 hours for 4 hours. FIG. 5A shows overlaid UV-VIS spectra of free avobenzone layered as a thin-film in a lotion base and irradiated for various times at 365 nm using a 100 W lamp located 14 cm from the sample. FIG. 5B shows overlaid UV-VIS spectra of AVB@MAF-6 layered as a thin-film in a lotion base and irradiated for various times at 365 nm using a 100 W lamp located 14 cm from the sample. As with the irradiations conducted in methanol, free avobenzone experienced photodegradation at a much more rapid rate than did the avobenzone in AVB@MAF-6. Whereas only approximately 25% of the free avobenzone remained after 4 hours of irradiation, approximately 70% remained when incorporated within the AVB@MAF-6. This experiment simulates sun-protection performance of the AVB@MAF-6 upon human skin.
I: Photostability of OMC@MAF-6 Dispersed in Methanol. Free octyl p-methoxycinnamate (OMC) and OMC@MAF-6 were dissolved or dispersed in methanol at equivalent loadings of OMC, and the samples were irradiated at 365 nm using a 100 W lamp located 14 cm from the sample. UV-VIS spectra were taken for both samples during the irradiation time period. Free OMC was approximately 30% photodegraded after 1 hour of irradiation (data not shown). FIG. 6 shows overlaid UV-VIS spectra of OMC@MAF-6 dispersed in methanol and irradiated for various times at 365 nm using a 100 W lamp located 14 cm from the sample. The photodegradation of OMC incorporated within the MOF significantly decreased relative to the free OMC. As shown, there was only approximately 4.7% photodegradation at 1 hour of irradiation, and at 4 hours of irradiation, the amount of photodegradation had risen to only approximately 15%.
J: Incorporation Stability of AVB@MAF-6. AVB@MAF-6 was dispersed in water at pH values of 5.5, 6.3, and 7.4 (these pH values correspond to those of blood, normal physiological pH, and sweat) and maintained under each of the various pH conditions for 4 hours. The supernatant liquid was removed by decantation and assayed by UV-VIS spectroscopy for the presence of avobenzone. FIG. 7 shows overlaid UV-VIS spectra of the supernatant liquids at various pH values obtained from AVB@MAF-6 in comparison to free avobenzone. As shown, based on the absorbance at the λₘₐₓ of free avobenzone, there was essentially no leaching at pH values of 6.3 and 7.4 and very minimal leaching at a pH value of 5.5.

### Example 2: Loading of β-CD-K-MOF with Adsorbates and Characterization Thereof

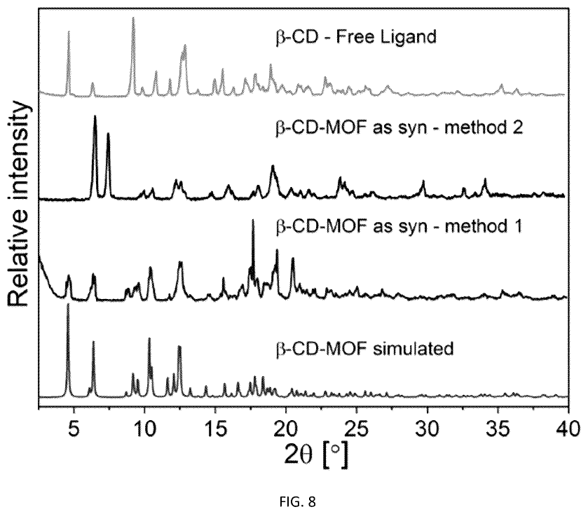

A: Synthesis of β-CD-K-MOF by Vapor Diffusion (KOH Method). The title MOF was synthesized by combining β-cyclodextrin and KOH in a 1:8 molar ratio in a glass vial containing 10 mL H₂O at 25° C. The glass vial was placed in a glass bottle containing 25 mL of methanol. The glass bottle was sealed with a screw cap, and the product was isolated following vapor diffusion-based crystallization for 5 days. The mean particle size was 1906±330 nm.
A1. Synthesis of β-CD-K-MOF by Solvent-Assisted Synthesis (KOH Method). β-cyclodextrin and KOH were combined in deionized water at a 1:8 molar ratio. The reaction mixture was then heated at 70° C. for 12 hours. After cooling to room temperature, the title MOF was obtained by precipitation, filtration, washing with methanol, and air drying.
A2. Synthesis of β-CD-K-MOF by Solvent-Assisted Synthesis (KNO₃ Method). The title MOF was synthesized by combining β-cyclodextrin and KOH in H₂O and heating at 70° C. The product was isolated following precipitation. The mean particle size was 529±69 nm. The mean particle size was reduced further to 231±65 following milling for 1 hour. FIG. 8 is an X-Ray powder diffraction pattern of β-CD-K-MOF synthesized using the alternative sources of potassium ions.

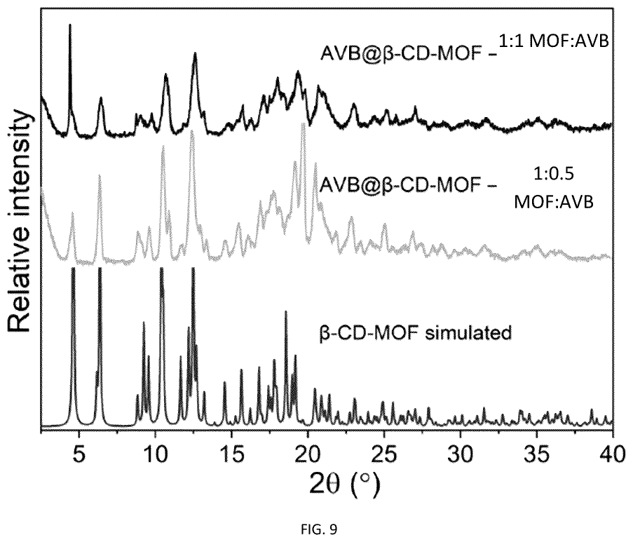

The synthesis of β-CD-K-MOF may also be conducted in a similar manner using different anions in combination with potassium. KF, KCl, KBr, KI, K₂H₂PO₄, KHPO₄, and K₂CO₃ all produced crystalline MOF phases. Of these, the β-CD-K-MOF phase produced from KF demonstrated the greatest uptake of avobenzone (approximately 40 wt %).
B: Loading of β-CD-K-MOF with Avobenzone (AVB@β-CD-K-MOF). β-CD-K-MOF (KOH synthesis) and avobenzone (AVB) were combined and milled together without solvent for 1 hour. At a MOF: AVB mass ratio of 1:0.5, the loading of avobenzone within the MOF was 11.45 wt %, based upon mass of the MOF and as determined by UV-VIS spectroscopy. At a MOF:AVB mass ratio of 1:1, the loading of avobenzone within the MOF was 25.48 wt %, based upon mass of the MOF and as determined by UV-VIS spectroscopy. FIG. 9 is an X-Ray powder diffraction pattern of AVB@β-CD-K-MOF prepared under various conditions.

By using an Attritor Mill for 1 hour, the loading of avobenzone in the β-CD-K-MOF increased to 35 wt %, based upon mass of the MOF and as determined by UV-VIS spectroscopy.

Sonication of the as-produced AVB@β-CD-K-MOF in ethanol for 10, 20, and 40 minutes reduced the particle size to 790±12 nm, 570±95 nm, and 489±90 nm, respectively, as measured by dynamic light scattering. Measurement of particle size by SEM following sonication showed a particle size of 275±11 nm. The larger particle size observed by dynamic light scattering is believed to result from agglomeration-based skewing the dynamic light scattering data.

Loading of avobenzone in β-CD-K-MOF (KNO₃ synthesis) was conducted in a similar manner to that of β-CD-K-MOF (KOH synthesis). After milling the 231 nm β-CD-K-MOF (KOH synthesis) with avobenzone for 1 hour, the avobenzone was incorporated in the AVB-β-CD-K-MOF at 35 wt %, based upon total mass of the MOF and as determined by UV-VIS spectroscopy.
C: Loading of β-CD-K-MOF with Octyl p-methoxycinnamate (OMC@β-CD-K-MOF). β-CD-K-MOF and octyl p-methoxycinnamate (OMC) were combined at a 1:1 mass ratio and milled together without solvent for 1 hour. The loading of OMC within the MOF was 2.68 wt %, based upon total mass of the MOF and as determined by UV-VIS spectroscopy.

The loading of OMC was poor under solvent-assisted conditions using methanol as a solvent. However, when OMC was used as the solvent and heated neat with the β-CD-K-MOF at 80° C., 37 wt % loading of the OMC within the MOF was realized.

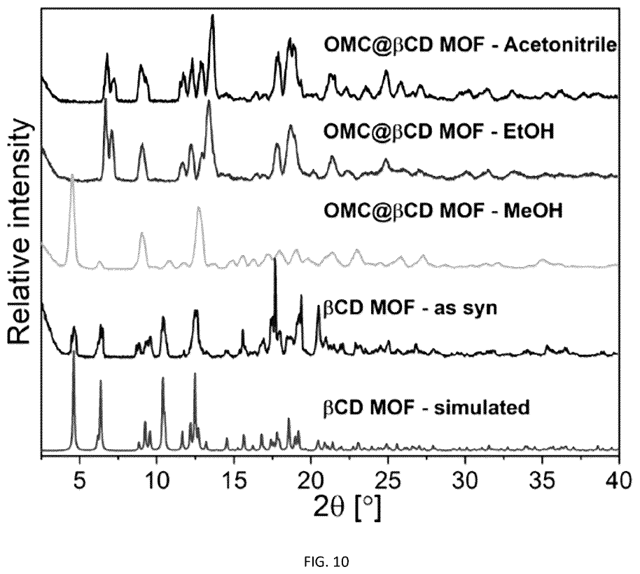

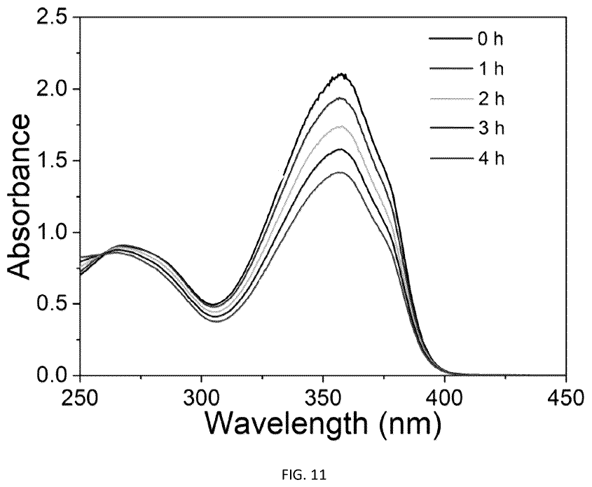

Loading of β-CD-K-MOF with OMC was repeated at 70° C. using acetonitrile, ethanol, or methanol as the solvent, which resulted in OMC loadings within the MOF of 2.67 wt %, 1.56 wt %, and 0.94 wt %, respectively, based upon total mass of the MOF and as determined by UV-VIS spectroscopy. Thus, using different solvents for solvent-assisted loading of OMC within the β-CD-K-MOF resulted in only minimally improved or poorer incorporation of the adsorbate within the MOF. FIG. 10 is an X-Ray powder diffraction pattern of OMC@)β-CD-K-MOF prepared under various solvent-assisted conditions.
D: Loading of β-CD-K-MOF with Avobenzone and Octyl p-methoxycinnamate (AVB/OMC@β-CD-K-MOF). β-CD-K-MOF, avobenzone, and octyl p-methoxycinnamate were combined in a 1:1:1 mass ratio and milled together without solvent. The loadings of avobenzone and octyl p-methoxycinnamate within the MOF were 0.75 wt % and 1.24 wt %, respectively, based upon total mass of the MOF and as determined by UV-VIS spectroscopy.
E: Photostability of AVB@β-CD-K-MOF Dispersed in Methanol. Free avobenzone and AVB-β-CD-K-MOF (KOH synthesis) were dissolved or dispersed in methanol at equivalent loadings of avobenzone, and the samples were irradiated with 365 nm UV radiation using a 100 W lamp located 14 cm from the sample. UV-VIS spectra were taken for both samples during the irradiation time period. As in Example 1, free avobenzone underwent essentially full photodegradation within 2 hours of irradiation (see FIG. 2A). FIG. 11 shows overlaid UV-VIS spectra of AVB@ β-CD-K-MOF dispersed in methanol and irradiated at 365 nm for various irradiation times using a 100 W lamp located 14 cm from the sample. After 4 hours of irradiation, the avobenzone was 32% photodegraded when incorporated within the β-CD-K-MOF.

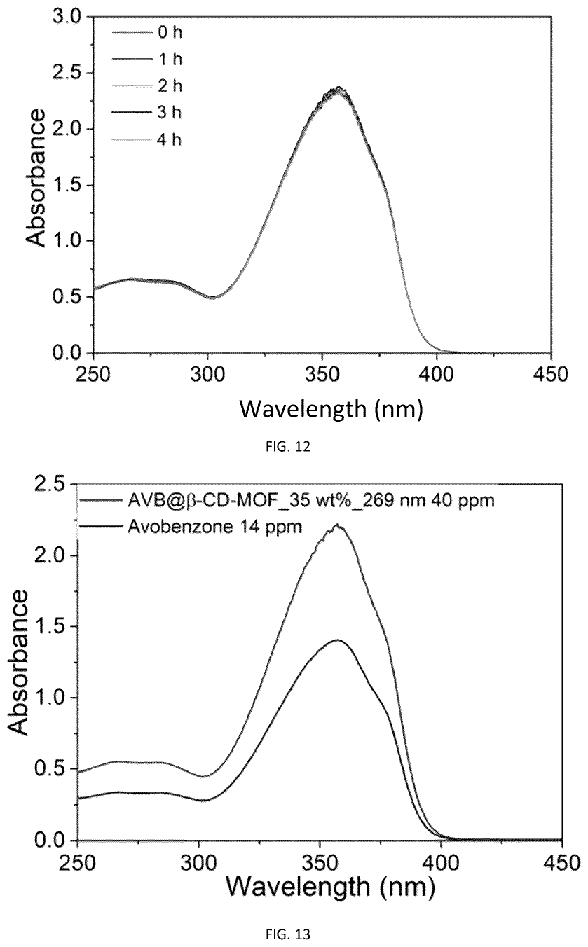

Photostability of avobenzone was also assessed in AVB@β-CD-K-MOF having smaller particle sizes. Surprisingly, increased photostability of avobenzone was realized at the smaller particle sizes. FIG. 12 shows overlaid UV-VIS spectra of AVB@β-CD-K-MOF having an average particle size of 269 nm dispersed in methanol and irradiated for various times at 365 nm using a 100 W lamp located 14 cm from the sample. In comparison to AVB@β-CD-K-MOF having larger particle sizes (see FIG. 11, for example), a considerably lower degree of avobenzone photodegradation was observed. Whereas greater than 30% of the avobenzone in larger AVB@β-CD-K-MOF particles photodegraded within 4 hours, only 4% photodegradation occurred in the 269 nm AVB@β-CD-K-MOF particles.

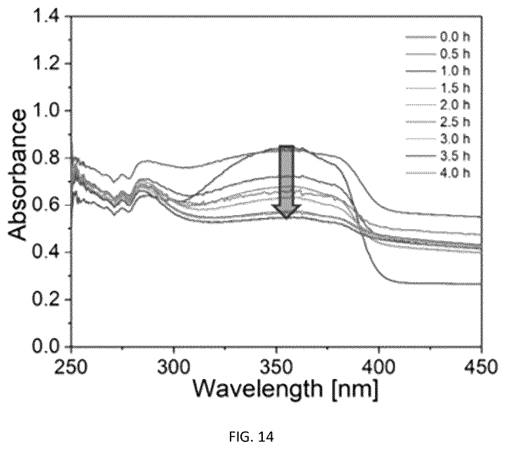

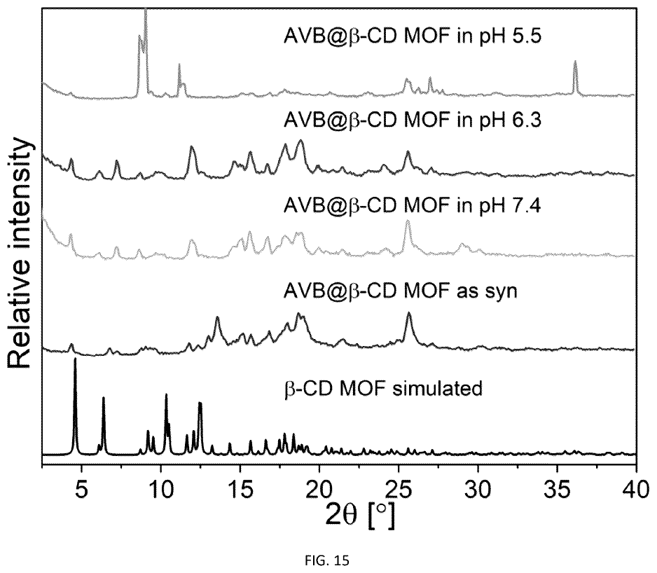

Additionally, at smaller AVB@β-CD-K-MOF particle sizes, increased UV absorbance for avobenzone was realized relative to an equivalent concentration of free avobenzone. FIG. 13 shows overlaid UV-VIS spectra for AVB@β-CD-K-MOF and free avobenzone at equivalent avobenzone concentrations. As shown, the encapsulated avobenzone displayed approximately a 1.6-fold increase in absorbance relative to free avobenzone.
F: Photostability of AVB@β-CD-K-MOF as a Thin-Film. Avobenzone and AVB@β-CD-K-MOF were separately formulated in a lotion base, such that the avobenzone concentration in the lotion base was 3 wt %, based on total mass. 32.5 mg of each formulation was spread uniformly upon a PMMA plate to provide a thin-film. The thin-films were then irradiated for various times at 365 nm using a 100 W lamp located 14 cm from the sample. UV-VIS spectra were acquired every 0.5 hours for 4 hours. FIG. 14 shows overlaid UV-VIS spectra of AVB@β-CD-K-MOF layered as a thin-film in a lotion base and irradiated for various times at 365 nm using a 100 W lamp located 14 cm from the sample. As with the irradiations conducted in methanol, free avobenzone (FIG. 5A) experienced photodegradation at a much more rapid rate than did the avobenzone in AVB@β-CD-K-MOF. Whereas only 25% of the free avobenzone remained after 4 hours of irradiation, approximately 65% remained when incorporated within the AVB@β-CD-K-MOF. The photodegradation of avobenzone was not impacted when octyl p-methoxycinnamate (not in a MOF) or octocrylene (not in a MOF) was blended with the lotion base in combination with the AVB@β-CD-K-MOF.
G: Incorporation Stability of Avb@β-CD-K-MOF. Avb@β-CD-K-MOF Was dispersed in water at pH values of 5.5, 6.3, and 7.4 (these pH values correspond to those of blood, normal physiological pH, and sweat) and maintained under each of the various pH conditions for 4 hours. The supernatant liquid was removed by decantation and assayed by UV-VIS spectroscopy for the presence of avobenzone. The UV-VIS spectra of the supernatant liquid (not shown) showed no more than 1 wt % leaching of the avobenzone from the MOF, as evaluated based upon the absorbance at λₘₐₓ of the avobenzone. FIG. 15 shows X-Ray powder diffraction patterns of the AVB@β-CD-K-MOF treated at the various pH values. As shown, the crystalline phase of the samples treated at pH values of 6.3 and 7.4 were essentially unchanged from that of the untreated MOF. In contrast, a crystalline phase change was observed in the sample treated at a pH value of 5.5. The crystalline phase change was not observed when similarly treating smaller particles of AVB@β-CD-K-MOF.

### Example 3: Loading of HP-β-CD-K-MOF with Adsorbates and Characterization Thereof

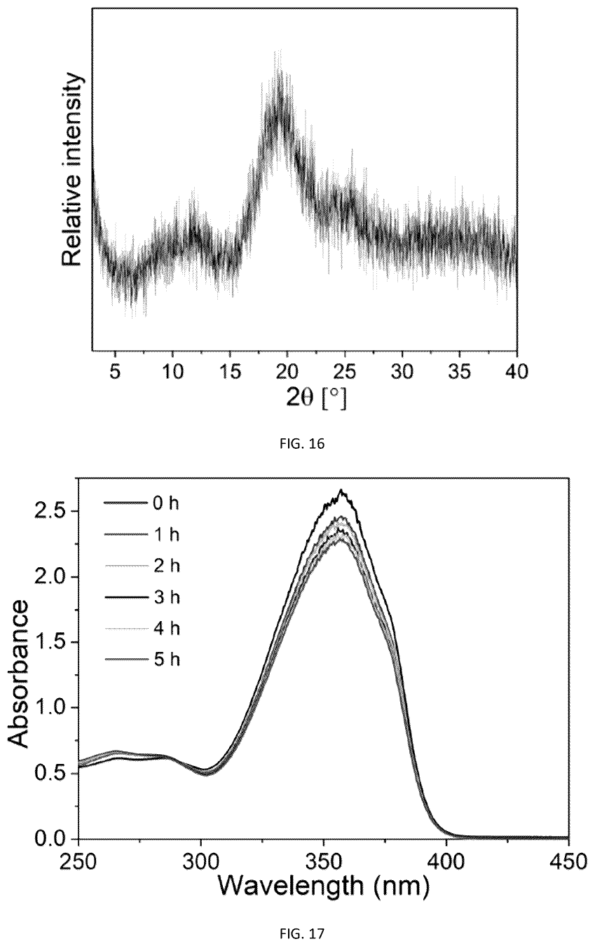

A: Synthesis of HP-β-CD-K-MOF. The title MOF was synthesized by combining 2-hydroxypropyl-β-cyclodextrin (0.137 mmol) and KOH in deionized water at a 1:8 molar ratio. The reaction mixture was heated at 70° C. for 12 hours. After cooling to room temperature, acetone was combined with the reaction mixture to rapidly precipitate the product as an amorphous white solid. The product was isolated by filtration, washed with acetone, and air dried. Additional characterization of the product was performed by ¹H NMR spectroscopy and infrared spectroscopy (neither shown). The mean particle size was 1618±264 nm. FIG. 16 is an X-Ray powder diffraction pattern of HP-β-CD-K-MOF.
B. Loading of HP-β-CD-K-MOF with Avobenzone (AVB@HP-β-CD-K-MOF). HP-β-CD-K-MOF (KOH synthesis) and avobenzone (AVB) were combined at a 1:1 mass ratio and milled together without solvent for 1 hour. The loading of avobenzone within the MOF was 21.28 wt %, based upon total mass of the MOF and as determined by UV-VIS spectroscopy.
C. Loading of HP-β-CD-K-MOF with Octyl p-methoxycinnamate (OMC@HP-β-CD-K-MOF). HP-β-CD-K-MOF and octyl p-methoxycinnamate (OMC) were milled together without solvent for 1 hour. The loading of OMC within the MOF was 1.67 wt %, based upon total mass of the MOF and as determined by UV-VIS spectroscopy. Solvent-assisted loading of OMC afforded 20 wt % incorporation of the adsorbate within the MOF.
D. Loading of HP-β-CD-K-MOF with Avobenzone and Octyl p-methoxycinnamate (AVB/OMC@HP-β-CD-K-MOF). HP-β-CD-K-MOF, avobenzone, and octyl p-methoxycinnamate were milled together without solvent for 1 hour. The loadings of avobenzone and octyl p-methoxycinnamate within the MOF were 0.59 wt % and 1.42 wt %, respectively, based upon total mass of the MOF and as determined by UV-VIS spectroscopy.

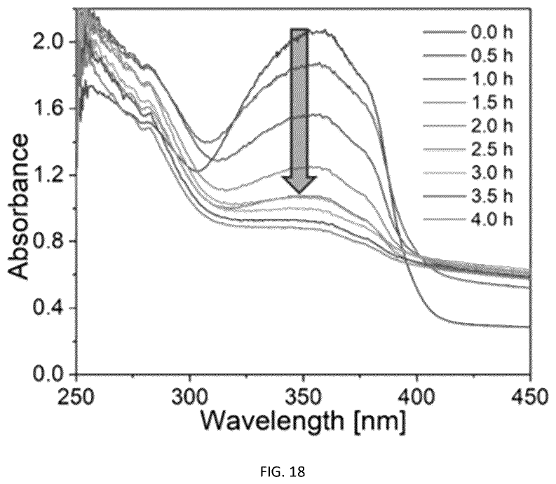

Solvent-assisted loading of avobenzone and OMC in equal amounts afforded 1 wt % incorporation of avobenzone within the MOF and 8 wt % incorporation of the OMC within the MOF.
E: Photostability of AVB@HP-β-CD-K-MOF in Methanol. Free avobenzone and HP-β-CD-K-MOF were dissolved or dispersed in methanol at equivalent loadings of avobenzone, and the samples were irradiated at 365 nm using a 100 W lamp located 14 cm from the sample. UV-VIS spectra were taken for both samples during the irradiation time period. As discussed above, free avobenzone (FIG. 2A) underwent essentially full photodegradation within 2 hours of irradiation. FIG. 17 shows overlaid UV-VIS spectra of AVB@HP-β-CD-K-MOF dispersed in methanol and irradiated for various irradiation times at 365 nm using a 100 W lamp located 14 cm from the sample. After 4 hours of irradiation, only 13 wt % of the avobenzone incorporated within the HP-β-CD-K-MOF had undergone photodegradation.
F: Photostability of AVB@HP-β-CD-K-MOF as a Thin-Film. Avobenzone and AVB@HP-β-CD-K-MOF were separately formulated in a lotion base, such that the avobenzone concentration in the lotion base was 3 wt %, based on total mass. 32.5 mg of each formulation was spread uniformly upon a PMMA plate to provide a thin-film. The thin-films were then irradiated for various times at 365 nm using a 100 W lamp located 14 cm from the sample. UV-VIS spectra were acquired every 0.5 hours for 4 hours. FIG. 18 shows overlaid UV-VIS spectra of AVB@HP-β-CD-K-MOF layered as a thin-film in a lotion base and irradiated for various times at 365 nm using a 100 W lamp located 14 cm from the sample. As with the irradiations conducted in methanol, free avobenzone (FIG. 5A) experienced photodegradation at a much more rapid rate than did the avobenzone in AVB@ HP-β-CD-K-MOF. Whereas only 25% of the free avobenzone remained after 4 hours of irradiation, approximately 45% remained when incorporated within the AVB@HP-β-CD-K-MOF.

### Example 4: Loading of γ-CD-K-MOFs with Adsorbates and Characterization Thereof

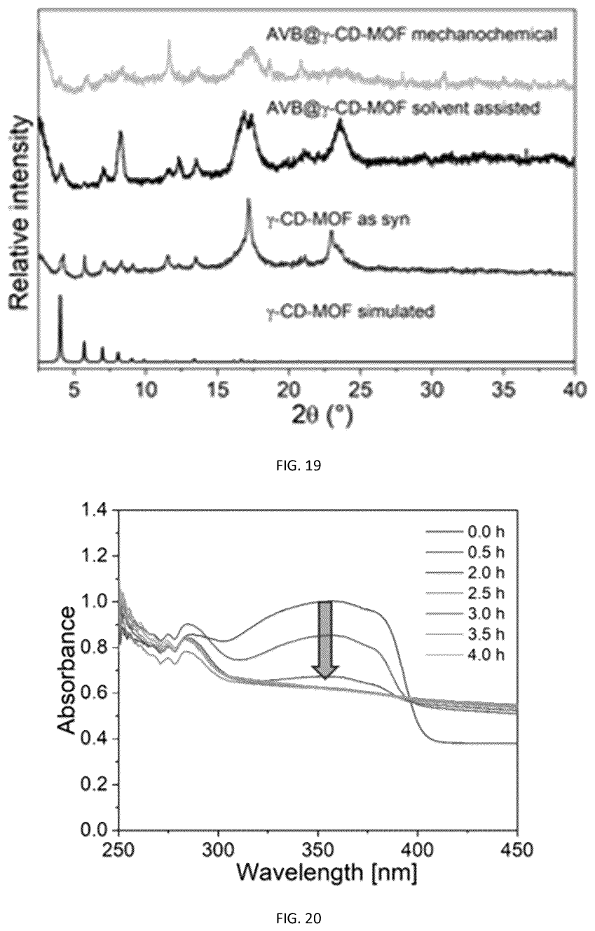

A. Synthesis of γ-CD-K-MOFs. γ-cyclodextrin and KOH were combined in a 1:8 molar ratio and placed into a 20 mL glass vial containing 10 mL of deionized water. The glass vial was then placed in a glass bottle containing 25 mL of MeOH. The glass bottle was sealed with a screw cap to allow for the diffusion of methanol into the reaction mixture. After 5 days, the crystallized γ-CD-K-MOF was isolated.
B. Loading of γ-CD-K-MOF with Avobenzone (AVB@γ-CD-K-MOF). γ-CD-K-MOF and avobenzone (AVB) were combined and milled together for 1 hour. The loading of avobenzone within the MOF was approximately 20 wt %, based upon total mass of the MOF and as determined by UV-VIS spectroscopy. FIG. 19 shows overlaid X-Ray powder diffraction spectra for γ-CD-K-MOF and AVB@γ-CD-K-MOF.
C: Photostability of AVB@γ-CD-K-MOF as a Thin-Film. Avobenzone and AVB@γ-CD-K-MOF were separately formulated in a lotion base, such that the avobenzone concentration in the lotion base was 3 wt %, based on total mass. 32.5 mg of each formulation was spread uniformly upon a PMMA plate to provide a thin-film. The thin-films were then irradiated for various times at 365 nm using a 100 W lamp located 14 cm from the sample. UV-VIS spectra were acquired every 0.5 hours for 4 hours. FIG. 20 shows overlaid UV-VIS spectra of AVB@γ-CD-K-MOF layered as a thin-film in a lotion base and irradiated for various times at 365 nm using a 100 W lamp located 14 cm from the sample. As with the irradiations conducted in methanol, free avobenzone (FIG. 5A) experienced photodegradation at a much more rapid rate than did the avobenzone in AVB@γ-CD-K-MOF. Whereas only 25% of the free avobenzone remained after 4 hours of irradiation, approximately 67% remained when incorporated within the AVB@γ-CD-K-MOF.

### Example 5: Loading of ZIF-8 with Adsorbates and Characterization Thereof

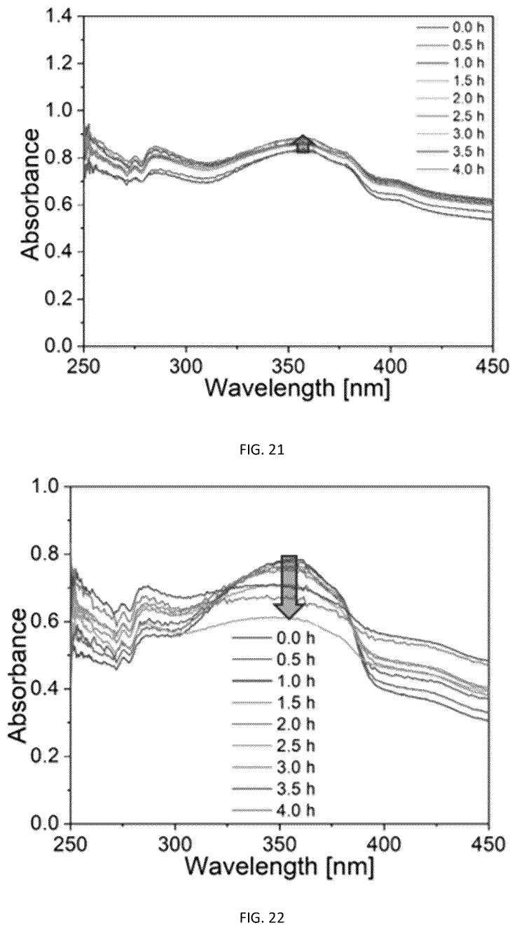

A 1. Synthesis of ZIF-8. A solid mixture of 1 mmol of zinc oxide, 3 mmol of 2-methylimidazole, and 200μL of solvent was placed in a milling jar, and the mixture was milled for 1 hour. The resulting solids were washed with deionized water and alcohol and then dried in an oven at 80° C. for 12 hours.
A 2. Synthesis of ZIF-8 by Ultrasonication. 0.94 g of zinc nitrate hexahydrate and 3.3 g of 2-methylimidazole were separately dissolved in 20 mL of methanol. The two solutions were heated separately in a water bath at 30° C. The 2-methylimidazole solution was then quickly added to the zinc nitrate hexahydrate solution, and the combined mixture was ultrasonicated at 30° C. for 1 hour. A solid was obtained by centrifugation, which was then washed three times with methanol, followed by air drying and oven drying at 80° C. overnight.
B. Loading of ZIF-8 with Avobenzone (AVB@ZIF-8). Avobenzone and ZIF-8 were combined in methanol in equal amounts and heated at 70° C. overnight. The product was obtained by cooling, washing with methanol, air drying, and finally oven drying at 80° C. for 12 hours.
C: Photostability of AVB@ZIF-8 as a Thin-Film. Avobenzone and AVB@ZIF-8 were separately formulated in a lotion base, such that the avobenzone concentration in the lotion base was 3 wt %, based on total mass. 32.5 mg of each formulation was spread uniformly upon a PMMA plate to provide a thin-film. The thin-films were then irradiated for various times at 365 nm using a 100 W lamp located 14 cm from the sample. UV-VIS spectra were acquired every 0.5 hours for 4 hours. FIG. 21 shows overlaid UV-VIS spectra of AVB@ZIF-8 layered as a thin-film in a lotion base and irradiated for various times at 365 nm using a 100 W lamp located 14 cm from the sample. As with the irradiations conducted in methanol, free avobenzone (FIG. 5A) experienced photodegradation at a much more rapid rate than did the avobenzone in AVB@ZIF-8. No photodegradation of the avobenzone in AVB@ZIF-8 was evident by UV-VIS. Surprisingly, the absorption actually increased slightly during the irradiation period.

### Example 6: Loading of MIL-100(Fe) With Adsorbates and Characterization Thereof

A. Synthesis of MIL-100(Fe). MIL-100(Fe) was synthesized hydrothermally according to reported procedures by combining FeCl₃·6H₂O (2.7 g), 1,3,5-benzenetricarboxylic acid (1.41 g), nitric acid (3 mL), and water (30 mL), and at 150° C. for 12 hours. The resulting solid was collected and washed several times with deionized water and ethanol, soaking in ethanol, and then drying
B. Loading of MIL-100(Fe) with Avobenzone (AVB@MIL-100(Fe)). MIL-100(Fe) and avobenzone were milled together for 1 hour. The loading of avobenzone within the MOF was approximately 40 wt %, based upon total mass of the MOF and as determined by UV-VIS spectroscopy.
C: Photostability of Avb@MIL-100(fe) As a Thin-film. Avobenzone and AVB@MIL-100(Fe) were separately formulated in a lotion base, such that the avobenzone concentration in the lotion base was 3 wt %, based on total mass. 32.5 mg of each formulation was spread uniformly upon a PMMA plate to provide a thin-film. The thin-films were then irradiated for various times at 365 nm using a 100 W lamp located 14 cm from the sample. UV-VIS spectra were acquired every 0.5 hours for 4 hours. FIG. 22 shows overlaid UV-VIS spectra of AVB@MIL-100(Fe) layered as a thin-film in a lotion base and irradiated for various times at 365 nm using a 100 W lamp located 14 cm from the sample. As with the irradiations conducted in methanol, free avobenzone (FIG. 5A) experienced photodegradation at a much more rapid rate than did the avobenzone in AVB@MIL-100(Fe). Whereas only 25% of the free avobenzone remained after 4 hours of irradiation, approximately 78% remained when incorporated within the AVB@MIL-100(Fe).

### Example 7: Loading of UiO-66-NH₂ with Adsorbates and Characterization Thereof

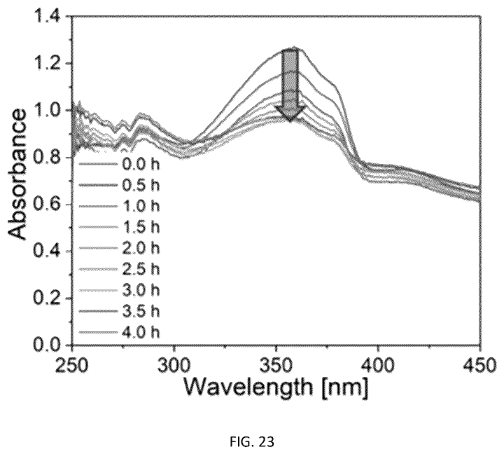

A. Synthesis of UiO-66-NH₂. UiO-66-NH₂ was synthesized according to reported procedures by combining ZrCl₄ (4.66 g), DMF (560 mL), concentrated HCl (37 mL), and 2-aminoterephthalic acid (5 g), sonicating until fully dissolved, and heating at 80° C. overnight. The resulting solid was collected, washed with DMF and then ethanol, and finally air dried to afford a pale yellow solid.
B. Loading of UiO-66-NH₂ with Avobenzone (AVB@UiO-66-NH₂). 1 g of avobenzone and 1 g of UiO-66-NH₂ were combined in 200 mL of methanol and heated at 70° C. overnight. The product was obtained by cooling, washing with methanol, air drying, and finally oven drying at 80° C. for 12 hours.
C: Photostability of AVB@ UiO-66-NH₂ as a Thin-Film. Avobenzone and AVB@UiO-66-NH₂ were separately formulated in a lotion base, such that the avobenzone concentration in the lotion base was 3 wt %, based on total mass. 32.5 mg of each formulation was spread uniformly upon a PMMA plate to provide a thin-film. The thin-films were then irradiated for various times at 365 nm using a 100 W lamp located 14 cm from the sample. UV-VIS spectra were acquired every 0.5 hours for 4 hours. FIG. 23 shows overlaid UV-VIS spectra of AVB@UiO-66-NH₂ layered as a thin-film in a lotion base and irradiated for various times at 365 nm using a 100 W lamp located 14 cm from the sample. As with the irradiations conducted in methanol, free avobenzone (FIG. 5A) experienced photodegradation at a much more rapid rate than did the avobenzone in AVB@UiO-66-NH₂. Whereas only 25% of the free avobenzone remained after 4 hours of irradiation, approximately 75% remained when incorporated within the AVB@UiO-66-NH₂.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element, or group of elements is preceded with the transitional phrase “comprising,” it is understood that we also contemplate the same composition or group of elements with transitional phrases “consisting essentially of,” “consisting of,” “selected from the group consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

One or more illustrative embodiments are presented herein. Not all features of a physical implementation are described or shown in this application for the sake of clarity. It is understood that in the development of a physical embodiment of the present disclosure, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for one of ordinary skill in the art and having benefit of this disclosure.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to one having ordinary skill in the art and having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present disclosure. The embodiments illustratively disclosed herein suitably may be practiced in the absence of any element that is not specifically disclosed herein and/or any optional element disclosed herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention. What is claimed is the following:

1. A metal-organic framework comprising hydrophobic cavities having a pore diameter of about 1 nm to about 2 nm, and, optionally, at least one adsorbate within the hydrophobic cavities.

2. The metal-organic framework of claim 1, wherein the hydrophobic cavities have an aperture size of about 0.76 nm.

3. The metal-organic framework of claim 1, wherein the metal-organic framework comprises a zeolitic imidazolate having RHO topology and cuboctahedral hydrophobic cavities of about 2 nm pore diameter.

4. The metal-organic framework of claim 3, wherein the metal-organic framework has an average particle size of about 50 nm to about 2 microns.

5. The metal-organic framework of claim 3, wherein the metal-organic framework has an average particle size of about 60 nm to about 1 micron.

6. The metal-organic framework of claim 3, wherein the metal-organic framework has an average particle size of about 250 nm to about 350 nm.

7. The metal-organic framework of claim 3, wherein the metal-organic framework has an average particle size of about 125 nm to about 150 nm.

8. The metal-organic framework of claim 3, wherein the metal-organic framework has a BET surface area of about 1000 m²/gm to about 2000 m²/gm.

9. The metal-organic framework of claim 3, wherein the metal-organic framework has a BET surface area of about 1485 m²/gm.

10. The metal-organic framework of claim 3, wherein the metal-organic framework has an adsorption capacity of up to about 50 wt %, based on mass of the metal-organic framework.

11. The metal-organic framework of claim 3, wherein the metal-organic framework has an adsorption capacity of up to about 25 wt %, based on mass of the metal-organic framework.

12. The metal-organic framework of claim 3, wherein the at least one adsorbate is present.

13. The metal-organic framework of claim 12, wherein the at least one adsorbate comprises at least one hydrophobic adsorbate.

14. The metal-organic framework of claim 13, wherein the at least one hydrophobic adsorbate comprises at least one retinol adsorbate.

15. The metal-organic framework of claim 13, wherein the at least one hydrophobic adsorbate comprises at least one of avobenzone and octyl p-methoxycinnamate.

16. The metal-organic framework of claim 15, wherein the avobenzone, when incorporated within the metal-organic framework, exhibits less than 35% photodegradation when subjected to UV-A radiation for up to 8 hours.

17. The metal-organic framework of claim 15, wherein the avobenzone, when incorporated within the metal-organic framework, exhibits less than 30% photodegradation when subjected to UV-A radiation for up to 4 hours.

18. The metal-organic framework of claim 15, wherein the avobenzone, when incorporated within the metal-organic framework, exhibits less than 25% photodegradation when subjected to UV-A radiation for up to 5 hours.

19. The metal-organic framework of claim 15, wherein the avobenzone, when incorporated within the metal-organic framework, exhibits less than 15% photodegradation when subjected to UV-A radiation for up to 2 hours.

20. The metal-organic framework of claim 15, wherein the octyl p-methoxycinnamate, when incorporated within the metal-organic framework, exhibits less than 25% photodegradation when subjected to UV-A radiation for up to 4 hours.

21. The metal-organic framework of claim 15, wherein the octyl p-methoxycinnamate, when incorporated within the metal-organic framework, exhibits less than 15% photodegradation when subjected to UV-A radiation for up to 1 hour.

22. The metal-organic framework of claim 12, wherein the hydrophobic adsorbate, when incorporated within the metal-organic framework, exhibits up to about 25% increased absorbance at λₘₐₓ as compared to an equivalent concentration of free hydrophobic adsorbate.

23. The metal-organic framework of claim 12, wherein the hydrophobic adsorbate, when incorporated within the metal-organic framework, exhibits up to about 10% increased absorbance at λₘₐₓ as compared to an equivalent concentration of free hydrophobic adsorbate.

24. A composition comprising:
a metal-organic framework having a plurality of pores defined therein; and
at least one hydrophobic adsorbate incorporated within the plurality of pores;
wherein the at least one hydrophobic adsorbate is present within the plurality of pores in an amount ranging from about 20 wt % to about 50 wt %, based upon mass of the metal-organic framework; and
wherein photodegradation of the hydrophobic adsorbate, when incorporated within the plurality of pores, is at least partially suppressed relative to free hydrophobic adsorbate not incorporated within the plurality of pores.

25. The composition of claim 24, wherein the pores have a pore diameter of about 1 nm to about 2 nm.

26. The composition of claim 24, wherein the metal-organic framework is MAF-6.

27. The composition of claim 24, wherein the metal-organic framework is ZIF-8.

28. The composition of claim 24, wherein the metal-organic framework is MIL-100(Fe).

29. The composition of claim 24, wherein the metal-organic framework is UiO-66-NH₂.

30. The composition of claim 24, wherein the at least one hydrophobic adsorbate comprises a UV-absorber.

31. The composition of claim 30, wherein the UV-absorber comprises avobenzone, octyl p-methoxycinnamate, or any combination thereof.

32. The composition of claim 30, wherein the UV-absorber comprises at least one retinol compound.

33. The composition of claim 31, wherein the metal-organic framework is MAF-6.

34. The composition of claim 33, wherein the MAF-6 has an average particle size of about 125 nm to about 350 nm.

35. A personal care composition comprising the composition of claim 24.
